(19) United States
(12) Patent Application Publication
(10) Pub. No.: US 2008/0045864 A1
(43) Pub. Date: Feb. 21, 2008

(54) Dynamic acoustic focusing utilizing time reversal

(75) Inventors: James V. Candy, Danville, CA (US); David H. Chambers, Livermore, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California.

(21) Appl. No.: 11/904,823

(22) Filed: Sep. 28, 2007

Related U.S. Application Data
(63) Continuation of application No. 10/661,249, filed on Sep. 11, 2003.
(60) Provisional application No. 60/410,575, filed on Sep. 12, 2002.
(60) Provisional application No. 60/410,676, filed on Sep. 13, 2002.

(51) Int. Cl. A61N 7/00

(52) U.S. Cl. 601/2

(57) ABSTRACT

Noninvasively focusing acoustical energy on a mass such as a tumor within tissue to reduce or eliminate the mass. The presence of the mass in the tissue is detected by applying acoustic energy to the substance. The mass is localized to determine its position. Temporal signatures are developed to drive the acoustical energy on the mass. Dynamic focusing of the acoustical energy on the mass to reduce or eliminate it is accomplished utilizing the temporal signatures

### CROSS-REFERENCE TO RELATED APPLICATIONS

This application is a continuation of prior application Ser. No. 10/661,249 filed Sep. 11, 2003, entitled “Dynamic Acoustic Focusing Utilizing Time Reversal”, which claims the benefit of U.S. Provisional Application No. 60/410,575, filed Sep. 12, 2002, and entitled, “Dynamic Acoustic for Noninvasive Treatment”, both of which are incorporated herein by this reference. Any disclaimer that may have occurred during the prosecution of the above-referenced application Ser. No. 10/661,249 is hereby expressly rescinded.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to acoustic focusing and more particularly to dynamic acoustic focusing for noninvasive treatment.

2. State of Technology

U.S. Pat. No. 6,176,839 issued Jan. 23, 2001 for method and system for treatment with acoustic shock waves issued to Michael Deluis and Reiner Schultheiss provides the following state of technology information, “Acoustic shock waves are used in medicine for various indications. It is known that tumors and bodily secretions, such as gallstones, can be destroyed by acoustic shock waves. It is also known that the formation of new bone tissue can be induced and promoted by shock waves. Finally, shock waves are also used for generated outside the body, to pass through body tissue to arrive at the target area and be focused on this area. Depending on the type of treatment, it is intended and desired that the shock waves act with a greater or lesser degree of effectiveness in the target area. The body tissue through which the shock waves pass on their way to the target area, however, should interact as little as possible with the shock waves, because such interaction can lead to undesirable damage to this body tissue. So far, damage to the body tissue located outside the target area has been minimized essentially by focusing the shock waves. The shock waves passing through the body tissue outside the target area thus have a relatively low energy density, whereas the density of the shock waves in the target areas increased by focusing.”

U.S. Pat. No. 6,390,995 for a method for using acoustic shock waves in the treatment of medical conditions issued May 21, 2002 to John A. Ogden and John F. Warlick provides the following state of technology information, “The use of energy wave forms for medical treatment of various bone pathologies is known in the art. For example, U.S. Pat. No. 4,530,360, issued on Jul. 23, 1985 to Duarte, teaches the use of ultrasound transducers, in direct contact with the skin of the patient, for transmitting ultrasound pulses to the site of the bone defect. Duarte teaches a nominal ultrasound frequency of 1.3 to 2.0 MHz, a pulse width range of 10 to 2000 microseconds, and a pulse rate varying between 100 and 1000 Hz Duarte maintains the ultrasound power level below 100 milliwatts per square centimeter, with treatments lasting no more than 20 minutes per day. Other devices utilize piezoelectric materials fastened adjacent to the pathological site on the patient's limb to produce ultrasonic energy in the vicinity of the bone pathology for administering therapy. Examples of such prior art references include U.S. Pat. Nos. 5,211,160, 5,259,384, and 5,309,898.

Clinicians have also utilized shock waves to treat various pathologies. Early approaches of using shock waves for medical treatment required immersing the patient in water and directing a shock wave, generated by an underwater spark discharge, at a solid site to be treated, such as a bone or kidney stone. When the shock wave hits the solid site, a liberation of energy from the change of acoustic impedance from water to the solid site produces pressure in the immediate vicinity of the site. For example, U.S. Pat. No. 4,905,671 to Senge et al., issued on Mar. 6, 1990, teaches a method applying acoustic shock waves to induce bone formation. Senge et al. teaches that the acoustical sound waves utilized by Duarte (and similar references) for treatment of bone have a generally damped sinusoidal waveform centered on ambient pressure. More specifically, Senge et al. teaches that the pressure of an acoustical sound wave utilized by Duarte rises regularly to a maximum value above ambient, falls regularly through ambient and on to a minimum value below ambient in a continued oscillation above and below ambient until complete damping occurs. Portions of the wave above ambient represent acoustic compression, while portions below ambient represent acoustic tension.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a method of noninvasively focusing acoustical energy on a mass within a substance to reduce or eliminate the mass. The presence of the mass in the substance is detected by applying acoustic energy to the substance. The mass is localized to determine its position within the substance. Temporal signatures are developed to drive the acoustical energy on the mass. Dynamic focusing of the acoustical energy on the mass in the substance to reduce or eliminate the mass is accomplished utilizing the temporal signatures. In one embodiment the dynamic focusing of the acoustical energy on the mass utilizes time reversal. In another embodiment, the focusing of acoustical energy on a mass utilizes modeling and time reversal. In another embodiment, the focusing of acoustical energy on a mass utilizes modeling.

In one embodiment, the present invention provides a method of treating tissue by noninvasively focusing acoustical energy on a mass within the tissue to reduce or eliminate the mass. The embodiment comprising the steps of detecting the presence of the mass in the tissue by applying acoustic energy to the tissue, localizing the mass to determine its position within the tissue, developing temporal signatures to drive the acoustical energy on the mass, and dynamically focusing the acoustical energy on the mass in the tissue utilizing the temporal signatures to reduce or eliminate the mass. In one embodiment, the step of dynamic focusing the acoustical energy on the mass utilizes time reversal. In another embodiment the step of dynamic focusing the acoustical energy on the mass utilizes modeling and time reversal. In another embodiment the step of dynamic focusing the acoustical energy on the mass utilizes modeling.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 is a conceptual illustration of a system constructed in accordance with the present invention.

FIG. 2 is a conceptual illustration of an ultrasonic focusing system 200 for noninvasive mass treatment.

FIG. 3 illustrates time reversal focusing by a flow diagram.

FIG. 4 illustrates another embodiment of a system of the present invention.

FIG. 5 is a diagram of Matched-Field Processing.

FIG. 6 shows iterative time-reversal techniques.

FIG. 7 provides an example of interactive model-based T/R focusing.

FIG. 8 provides an example of model-based iterative T/R focusing.

FIG. 9 shows a mass localization algorithm using global/local iterations.

FIG. 10 shows time-reversal eigen-decomposition techniques.

FIG. 11 is a conceptual illustration of a system for noninvasive mass treatment and evaluation.

### DETAILED DESCRIPTION OF THE INVENTION

Referring now to the drawings, to the following detailed description, and to incorporated materials; detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

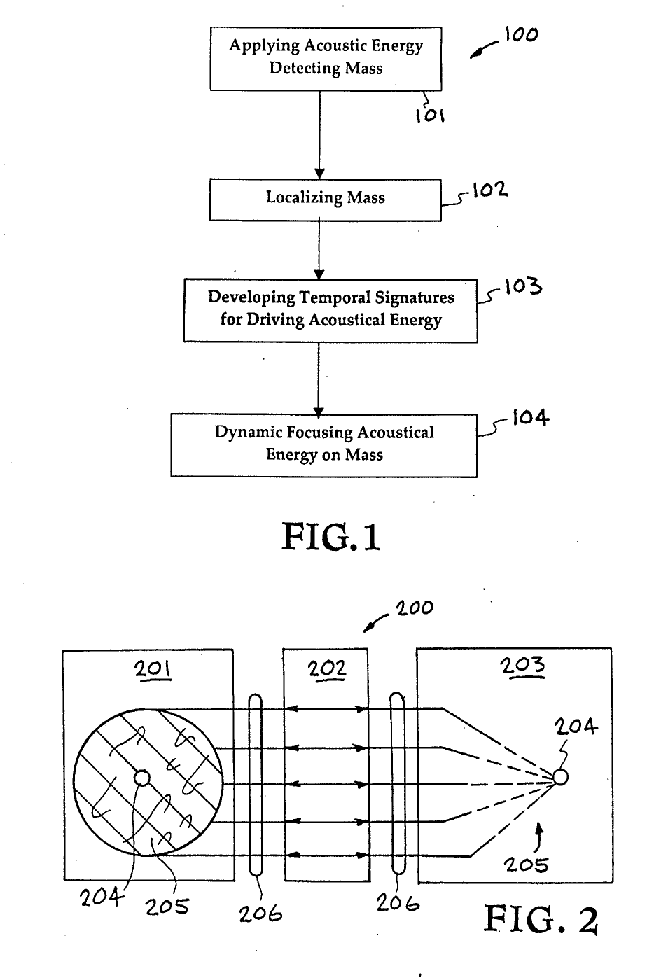

Referring now to the drawings and in particular to FIG. 1, a conceptual illustration of a system constructed in accordance with the present invention is illustrated. The system is designated generally by the reference numeral 100. The system provides methods and apparatus for noninvasively focusing acoustical energy on a mass within a substance to reduce or eliminate the mass. Acoustic energy is applied to the substance 101. The mass is localized 102 to determine its position within the substance. Temporal signatures are developed for driving acoustical energy on the mass 103. Dynamic focusing of acoustical energy on the mass 104 utilizing the temporal signatures reduces or eliminates the mass. In some embodiments the dynamic focusing of acoustical energy on the mass is accomplished utilizing time-reversal. In other embodiments the dynamic focusing of acoustical energy on the mass is accomplished utilizing modeling.

Methods of the system 100 comprise the steps of applying acoustic energy to the substance for detecting the presence of the mass in the substance 101, localizing the mass to determine its position within the substance 102, developing temporal signatures for driving the acoustical energy on the mass 103, and dynamically focusing the acoustical energy on the mass in the substance to reduce or eliminate the mass 104. In some embodiments the steps of developing temporal signatures and dynamic focusing are accomplished utilizing time-reversal. In other embodiments the steps of developing temporal signatures and dynamic focusing are accomplished utilizing modeling.

Apparatus of the system 100 comprise means 101 for transmitting an initial acoustic signal into the substance for detecting the mass, means 102 for localizing the mass, means 103 for developing temporal signatures for driving the acoustical energy, and means 104 for dynamically focusing the acoustical energy through the substance onto the mass to reduce or eliminate the mass. One embodiment of apparatus for implementing the method of the system 100 comprises a detector that transmits an initial acoustic signal into the substance, detects the mass, and produces an initial acoustic signal, a processor that digitizes the initial acoustic signal, a time-reversal processor that converts the initial acoustic signal that has been digitized into a time-reversal signal, and an acoustic energy device that uses the time-reversal signal and focuses the acoustical energy on the mass in the substance.

The dynamic focusing of acoustic energy is a technique that impacts a large number of applications ranging from noninvasively focusing acoustical energy on a mass within a substance to detecting and reducing or eliminating flaws in components. In the medical area, the system 100 has application in noninvasive tissue mass removal, non-invasive tumor/cyst destruction and treatment, and acoustic surgery. Treatment of tissue can be directly destructive through thermal or mechanical mechanisms, or indirectly destructive through localized enhancement of radiotherapy or chemotherapy caused by exposure to ultrasound. The system 100 has the prospect of opening new frontiers with the implication of noninvasive treatment of masses along with the expanding technology of acoustic surgery. The system 100 also has application in mass imaging, nondestructive evaluation of materials, secure communications, seismic detection of underground masses, and other applications.

In the system 100, the dynamic focusing 104 of acoustical energy on the mass utilizing the temporal signatures reduces or eliminates the mass. In some embodiments the dynamic focusing of acoustical energy on the mass is accomplished utilizing modeling. The modeling is described in detail below. In other embodiments the dynamic focusing of acoustical energy on the mass is accomplished utilizing time-reversal. Time-reversal techniques are described in detail in U.S. Pat. No. 6,490,469 for a method and apparatus for dynamic focusing of ultrasound energy issued Dec. 3, 2002 to James V. Candy and United States Patent Application No. 2003/0138053 for a time reversal communication system by James V. Candy and Alan W. Meyer published Jul. 24, 2003. The disclosures of U.S. Pat. No. 6,490,469 and United States Patent Application No. 2003/0138053 are incorporated herein by reference.

As illustrated in FIG. 1, the system 100 comprises a number of steps. The step 101 detects the presence of the mass in the substance by applying acoustic energy to the substance. Step 102 localizes the mass to determine its position within the substance. Step 103 develops temporal signatures to drive the acoustical energy on the mass. Step 104 provides dynamic focusing of the acoustical energy on the mass in the substance utilizing the temporal signatures thereby reducing or eliminating the mass. In one embodiment, the step 101 of detecting the presence of the mass in the substance comprises transmitting an initial acoustic signal into the substance for detecting the mass and detecting the initial acoustic signal. In one embodiment, the step 103 of developing temporal signatures to drive the acoustical energy on the mass comprises digitizing the initial acoustic signal and time-reversing the digitized initial acoustic signal. In one embodiment, the step 104 of dynamic focusing the acoustical energy on the mass in the substance comprises using the time-reversed initial acoustic signal in focusing the acoustical energy on the mass in the tissue. In one embodiment, the step 104 of dynamically focusing the acoustical energy on the mass in the substance comprises using modeling based upon the initial acoustic signal in focusing the acoustical energy on the mass in the tissue. In another embodiment, the step 101 of detecting the presence of the mass in the substance comprises applying acoustic energy propagated into the substance using an array of ultrasonic transducers. In another embodiment, the step 104 of dynamically focusing the acoustical energy on the mass in the substance utilizing time reversal generates heat and the heat essentially cooks the mass insuring reduction or elimination of the mass. In still another embodiment, the step 104 of dynamically focusing the acoustical energy on the mass in the substance utilizing time reversal creates mechanical disruption of cell membranes through cavitation and cell death. In another embodiment, the step 104 of dynamically focusing the acoustical energy on the mass in the substance utilizing time reversal induces a temporary increase of cell wall porosity to therapeutic agents, both chemical and genetic. In still another embodiment, the step 104 of dynamically focusing the acoustical energy on the mass in the substance utilizing time reversal ruptures microcapsules containing a therapeutic agent (chemical or genetic) for treatment of the mass.

The system 100 has the ability to noninvasively focus acoustical energy in tissue and directly on tissue masses such as tumors, cysts, etc. The system 100 provides the capability of focusing acoustic energy at a desired location for the purpose of treating tissue mass while minimizing the collateral damage in the surrounding tissue. When an ultrasonic wave is launched into tissue by a transducer or an array of transducers, the wave energy is absorbed, reflected or scattered by the tissue. The reflected/scattered energy received by a transducer represents the wave interaction with the tissue and is eventually used to create the image. The reflected energy received is due to changes in acoustic impedance across interfaces, while scattering occurs when the wave interacts with structures of size comparable to or less than an acoustic wavelength.

Probably the most critical issues in ultrasonic focusing are the acoustic characteristics of the tissue. The primary characteristics to consider are sound speed, attenuation, scattering, and inhomogeneities. Sound speed in soft tissue is approximately 1500 m/s, for instance, speeds in fat are about 1410 m/s, muscle is 1566 m/s, liver is 1540 m/s, while bone is 4080 m/s. Attenuation in different tissues increases in proportion to the excitation frequency. At 1 MHz fat, muscle, liver, and bone are: 0.63, 1.3-3.3, 0.94, 20 dB/cm. Typical ultrasonic designs attempt to operate at a high frequency in order to maximize spatial resolution, since frequency is inversely proportional to wavelength (above); however, as noted, attenuation increases with frequency thereby creating the tradeoff. The acoustic impedance (impedance=density×velocity) is directly related to sound speed at an interface, thereby, controlling the amplitude of the reflected/transmitted signals.

Again for these tissues (fat, muscle, liver, bone) the corresponding impedance is: 1.38, 1.7, 1.65, 7.8 10⁶ kg/m²-S. For instance, in the breast, which is dominated by fatty tissue, one of the major problems is scattering. An ultrasonic wave is scattered when it travels through tissue and the scattering pattern depends on the dimensions of the tissue structure in relation to the ultrasonic wavelength. Usually soft tissue is considered to be made up of many small scatterers which create noise in the image and must be processed to produce an enhanced image. So-called speckle noise is also a real artifact that must be reduced. Speckle is actually due to coherent illumination (and scattering) which can be reduced by broadband (in frequency) illumination. The inhomogeneity of biological tissue also distorts the ultrasonic wave because the differences in propagation speed create aberrations in the phase within the tissue. Thus, the design of an ultrasonic focusing system must take all of these factors into account and therefore presents a challenging technical problem.

Referring now to FIG. 2, a conceptual illustration of an ultrasonic focusing system 200 for noninvasive mass treatment is shown. The system is designated generally by the reference numeral 200. The system 200 comprises a “Detect/Localize” component 201, a “Time-Reversal” component 202, and a “Treatment” component 203. The system 200 has the ability to noninvasively focus acoustical energy 206 in tissue 205 and directly on a tissue mass 204 such as a tumor, a cyst, etc. The system 200 provides the capability of focusing acoustic energy 206 at a desired location for the purpose of treating a tissue mass 204 while minimizing the collateral damage in the surrounding tissue 205. This system 200 has the prospect of opening new frontiers with the implication of noninvasive treatment of tissue masses in the medical field along with the expanding technology of acoustic surgery.

The advent of high-speed digitizers, ultrafast computers, inexpensive memory, and the ability to construct dense acoustic arrays, the feasibility of noninvasive techniques of acoustic surgery offers an alternative to current invasive techniques. The focusing of acoustic energy to destructively treat a mass in surrounding tissue is an approach to noninvasive surgery. If the medium surrounding the mass is homogeneous it is a matter of focusing energy at a desired point in the medium. When the medium is inhomogeneous focusing at a desired focal point is more difficult unless some knowledge of the medium exists a-priori.

The system 200 provides the capability of focusing acoustic energy 206 at a desired location for the purpose of treating tissue mass while minimizing the collateral damage in the surrounding tissue. First, as illustrated by the Detect/Localize component 201, the presence of a tissue mass 204 is detected by applying acoustic energy 206 propagated into the tissue 205 using an array of ultrasonic transducers, time-reversal component 202. The amount of energy scattered by the mass 204 depends on its acoustic parameters (density, sound speed, attenuation, etc.). Once it is detected, the mass 204 is localized to determine its position within the tissue medium 205. Once detected and localized, temporal signatures are developed to “drive” the array, time-reversal component 202, and focus increased energy 206 back onto the mass 204 through the medium 205. The increased energy 206 generates heat, which essentially “cooks” the mass 204 insuring its destruction. Alternatively, the increased energy 206 can mechanically disrupt the tissue, enhance the porosity of cell membranes to therapeutic agents (chemical or genetic), or rupture microcapsules containing therapeutic agents.

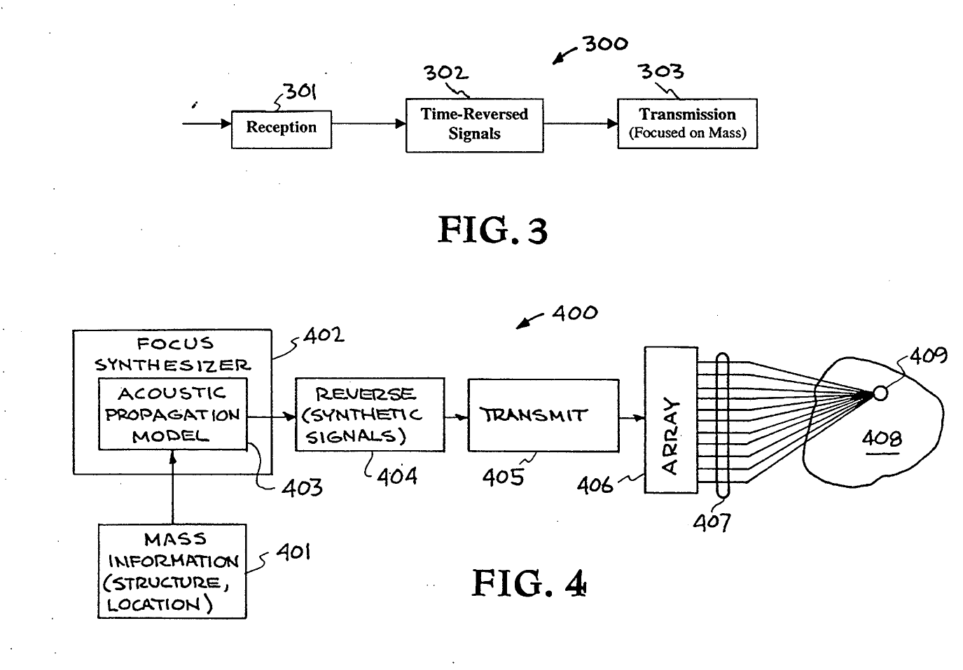

Referring now to FIG. 3, the time reversal focusing is illustrated by a flow diagram 300. After reception of scattered field, the temporal signals are reversed and retransmitted into the medium where the acoustic energy is focused on the mass. The flow diagram 300 shows reception 301, time-reversed signals 302, and transmission 303. When a source propagates through a spatio-temporal medium, the resulting wave front is distorted. If the medium is homogeneous and the source resides in the near field, then a spherical-type wave front evolves. But if the medium is inhomogeneous, then a distorted wave front results. In the first case, simple time-delay processing is sufficient to enhance the field at a given point; however, for inhomogeneous media the required time delays and amplitude are more difficult to estimate. The use of delay estimation and even adaptive delay estimation techniques become quite limited and unsuccessful in an inhomogeneous medium excited by a broadband incident field requiring an alternative approach to solve the focusing problem. The system utilizes “time-reversal processing” 300. The time-reversal processing 300 is applicable to spatio-temporal phenomena that satisfy a wave-type equation and possess a time reversal invariance property.

Dynamic focusing time reversal is essentially a technique to “focus” on a reflective target or mass through a homogeneous or inhomogeneous medium that is excited by a broadband source. More formally, time-reversal focusing converts a divergent wave generated from a source into a convergent wave focused on that source. Time reversal focusing can be thought of as an “optimal” spatio-temporal filter that adapts to the medium in which the wave front evolves and compensates for all geometric distortions while reducing the associated noise. The underlying theory and application of time-reversal techniques to acoustical problems have been developed along with a wide range of applications and proof-in-principle experiments. These applications have yielded some exciting results in focusing through an inhomogeneous medium and offer an opportunity for many different applications. This approach has been demonstrated for the focusing and destruction of painful kidney stones in lithotripsy. Fortunately, unlike tissue mass, the stones are highly reflective and the most dominant scatterer in the kidney.

Referring now to FIG. 4, another embodiment of a system of the present invention is illustrated. The system is designated generally by the reference numeral 400. The system 400 provides “Model-Based Focusing.” The system 400 includes providing mass information 401, a focus synthesizer 402, an acoustic propagation model 403, reverse (synthetic signals) 404, transmit 405, and a focus array 406. The acoustic energy 407 is transmitted through the medium to the tissue mass 408. The model-based approach develops a model of the inhomogeneous medium including the mass under scrutiny from the results of quantitative imaging, numerically propagates acoustic energy to the array 406 from a virtual source located at the mass generating a set of synthesized multichannel time series, and transmits the acoustic energy 407 back into the medium 408 to “focus” on the target mass 409.

“Blind” time reversal that will focus on the strongest scattering mass in a completely unknown tissue medium without any a-priori information about the medium, mass or its location is clearly a risky endeavor. In contrast, the model-based approach uses the model of the medium (including the mass and its location) to synthesize the appropriate time series and focus at the correct location. The major challenge of this approach is the development of the appropriate model. Quantitative imaging is applied using tomographic reconstruction techniques to characterize the medium model and an acoustic propagation algorithm to synthesize the required signals. In the system 400, after quantitative imaging, the propagation model is characterized, temporal signals are generated, reversed and transmitted into the medium where the acoustic energy is focused on the mass.

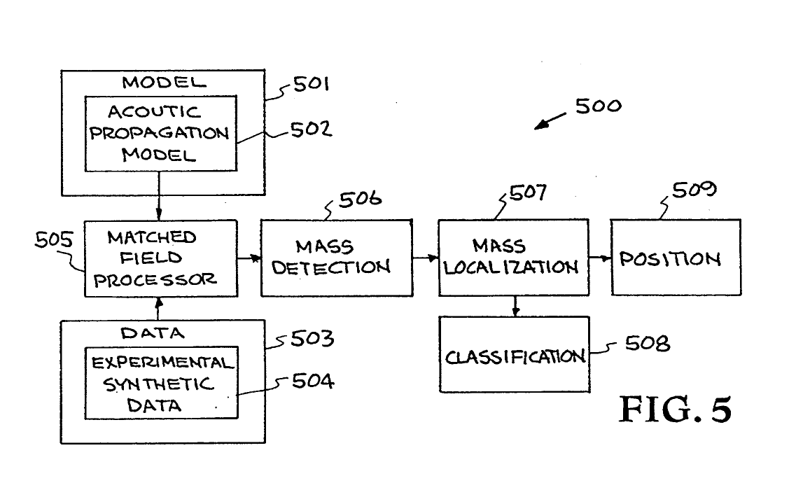

Referring now to FIG. 5, a diagram of Matched-Field Processing is shown. The matched-field processing is designated generally by the reference numeral 500. Matched-field processing 500 is considered by many to be an outgrowth of matched filtering in which a known signal such as a pulse in conventional ultrasound is transmitted into a medium and its return is to be detected from noisy measurements. A replicant of the pulse is convolved with the measurement to produce an optimal detection. When the pulse is unknown or cannot easily be measured or passive listening is assumed, then the replicant is no longer available and other methods must be used to generate the required replicant for optimal detection.

The system 500 uses a model 501 to produce an acoustic propagation model 502. Data 503 provides experimental synthetic data 504. The matched field processor 505 uses a propagation model 501 of the medium to generate the replicant for detection. Mass detection 506 and mass localization 507 provide classification 508 and position 509. The system 500 compares the model predicted field (replicant) propagated to the array position to the field actually measured at the sensor array to achieve the detection. In the localization problem, the matched-field processing 500 guesses at the position of a source, propagates it to the sensor array using the model 502 and compares it to the measured field. That location with the maximum power is deemed the location of the source. After careful preprocessing to remove extraneous signals and noise, the data are ready for imaging. Each pixel in the image representing a source or mass position is propagated to the sensor and its power or other feature is estimated to create the image. The threshold is applied to detect the presence of masses while their locations are determined by the corresponding maxima. Thus, in this way matched-field processing 500 offers a reasonable approach to imaging for mass detection and localization, when a propagation model is available.

Applicants begin their brief development of the processor with the overall field measured by a sensor or array of sensors and develop the basic signal models that will lead to a practical imaging technique. First, Applicants develop the underlying mathematical relationships to characterize their measured wave field.

Assume that the wave field resulting from the ultrasound satisfies the wave equation. The acoustic pressure at the l^{th}-sensor is given by
u(rₗ;t)=G(rₗ,rₛ;t)*s(rₛ;t), (1)
where

u(rₗ;t) is the ultrasonic wave field at the l^{th}-sensor; G(rₗ,rₛ;t) is the Green's function of the medium at rₗ,rₛ from the source-to-sensor at time t; and s(rₛ;t) is the source at rₛ and time t.

The actual sensor measurements are contaminated with gaussian random noise as well; therefore, Applicants define the noisy sensor measurement field as
zₗ(t)=u(rₗ;t)+nₗ(t), (2)
for nₗ the random noise contaminating the l-th sensor. If Applicants expand this expression over the entire L-element sensor array, then Applicants obtain the vector measurement field
z(t)=u(t)+n(t)=G(t)*s(rₛ,t)+n(t), (3)
where z,u, n,GεC^{L×1} are the measurement, field signal, white gaussian noise vector of variance σₙ²I, the medium Green's function and the respective source (mass) terms. Using this generic measurement model representing the noisy wave field measured across the array, Applicants next develop the matched-field (MF) processing approach.

The underlying problem is to decide whether or not there exists a mass in the tissue specimen. Assume that Applicants have the “known” replicant field signal, m(t), generated from their developed model (discussed above). Their problem is to detect a mass signal from the test specimen measurements. That is, Applicants must solve the binary decision problem
H₀: z(t)=n(t) [noise only]
H₁: z(t)=m(t)+n(t). [mass signal+noise] (4)
The solution to this problem is easily obtained from the Neyman-Pearson criterion and is given by the log-likelihood ratio test (LRT)

$$Λ ( \underline{z} ) = ln Pr ( \underline{z} \vert {H}_{1} ) - ln {Pr ( \underline{z} \vert {H}_{0} )}_{{<}_{{H}_{o}}}^{{>}^{{H}_{1}}} ln \tilde{λ} , \qquad ( 5 )$$

where Pr is the probability density function and \tilde{λ} is the threshold of the test. This problem, assuming that the measurements are zero-mean, gaussian with variance σₙ²I leads to the decision function

$$Λ ( \underline{z} ) = - {\frac{1}{2 {σ}_{n}^{2}} [ {( \underline{z} ( t ) - \underline{m} ( t ) )}^{′} ( \underline{z} ( t ) - \underline{m} ( t ) ) - {\underline{z}}^{′} ( t ) \underline{z} ( t ) ]}_{{<}_{{H}_{o}}}^{{>}^{{H}_{1}}} ln \tilde{λ} .$$

Expanding this expression and collecting all data dependent terms, Applicants obtain the sufficient statistic

$$Λ ( \underline{z} ) = {\underline{m}}^{′} ( t ) {\underline{z} ( t )}_{{<}_{{H}_{o}}}^{{>}^{{H}_{1}}} {σ}_{n}^{2} ln \tilde{ λ} + \frac{1}{2} {\underline{m}}^{′} ( t ) \underline{m} ( t ) ≡ λ . \qquad ( 6 )$$

Under the Neyman Pearson criterion, the threshold can be determined from the false alarm probability given by

$${P}_{FA} = {\int }_{λ}^{∞} Pr ( λ \vert {H}_{0} ) ⅆ λ$$

to a pre-selected value by solving for λ and \tilde{λ} in Eq. 6. In the white, gaussian noise case, Applicants have that Pr(λ|H₀)˜N(0,σₙ²I) which leads to the threshold [Joh93]

$$λ = \sqrt{{σ}_{n}^{2} E L} {Φ}^{- 1} ( {P}_{FA} ) \qquad ( 7 )$$

with the signal energy, E≡m′(t)m(t), Φ a unit variance gaussian distribution and L the number of sensors in the array.

Note also that by a simple change of variables in t, it is easy to show that the sufficient statistic of Eq. 6 is the well-known matched-filter solution with “matching” filter impulse response given in terms of their vector signal model of Eq. 6 by
m(t)≡u(T−t), and Λ(z)=u′(t−T)*z(t), (8)
which is simply the time reversed, replicant of the known field. Recall also from matched-filter theory that the desired solution is to find the optimal filter at each sensor channel such that the output signal-to-noise ratio (SNR) is maximized, that is, the matched-filter is the solution to

$${max}_{\underline{m}} S N R = \frac{{〈 {\underline{m}}^{′} ( T ) * \underline{z} ( T ) 〉}^{2}}{\frac{ {σ}_{ n}^{ 2}}{ 2} 〈 {\underline{m}}^{′} ( T ) * \underline{m} ( T ) 〉} = \frac{{〈 \int {\underline{m}}^{′} ( T - ξ ) \underline{z} ( ξ ) ⅆ ξ 〉}^{2}}{\frac{ {σ}_{ n}^{ 2}}{ 2} 〈 \int {\underline{m}}^{′} ( ξ ) \underline{m} ( ξ ) ⅆ ξ 〉} \qquad ( 9 )$$

for [symbol] • [symbol] an appropriate inner product yielding again
m(t)≡u(T−t). (10)

The important point here is that the matched-filter solution is simply the delayed, time reversed, replicant of the known field signal vector in the white, gaussian noise case. It is easy to extend this to the non-white noise case with the subsequent processor incorporating a pre-whitening filter (inverse of the noise covariance matrix) operation followed by the processor developed above.

In their solution, Applicants have assumed that the field vector, u(t), is completely known a priori. Suppose that the assumption is no longer true and Applicants can characterize the unknown or missing parameters (e.g. amplitude, phase, etc.) by the embedded vector, θ, then their field vector becomes u(t;θ) and therefore the “matching” vector is m(t;θ). The solution to this mass detection problem can be solved by composite hypothesis testing. In this case the test is
H₀: z(t)=n(t)
H₁: z(t)=m(t;θ)+n(t) (11)
with corresponding log-likelihood ratio

$$Λ ( \underline{z} ; \underline{θ} ) = ln Pr ( \underline{z} \vert \underline{θ} , {H}_{1} ) - ln {Pr ( \underline{z} \vert \underline{θ} , {H}_{0} )}_{{<}_{{H}_{o}}}^{{>}^{{H}_{1}}} ln {\tilde{λ}}_{θ} .$$

One solution to this problem is to estimate the parameter vector, \hat{θ} and then proceed as before which leads to the generalized log-likelihood ratio test (GLRT)

$${max}_{\underline{θ}} Λ ( \underline{z} ; \underline{θ} ) = {max}_{\underline{θ}} [ ln Pr ( \underline{z} \vert \underline{θ} , {H}_{1} ) ] - {max}_{\underline{θ}} {[ ln Pr ( \underline{z} \vert \underline{θ} , {H}_{0} ) ]}_{{<}_{{H}_{o}}}^{{>}^{{H}_{1}}} ln {\tilde{λ}}_{θ} . \qquad ( 12 )$$

Substituting m(t;θ)→m(t) in the previous relations, Applicants have that

$$Λ ( \underline{z} ; \underline{θ} ) = {\underline{m}}^{′} ( t ; \underline{θ} ) {z ( t )}_{{<}_{{H}_{o}}}^{{>}^{{H}_{1}}} {σ}_{n}^{2} ln {\hat{λ}}_{θ} + \frac{1}{2} {\underline{m}}^{′} ( t ; \underline{θ} ) \underline{m} ( t ; \underline{θ} ) ≡ {λ}_{θ} . \qquad ( 13 )$$

The result implies that as Applicants develop a solution to the mass detection problem, Applicants must search over the unknown parameter set, {θ} to maximize the log-likelihood using the GLRT to “match” the model replicant field to the data measured across the sensor array. This approach then leads to matched-field detection. Applicants search various parameter vectors and find that value θ that leads to the maximum log-likelihood or equivalent maximum output SNR power defined by

$${max}_{\underline{θ}} P ( \underline{θ} ) = {\frac{{〈 \int {\underline{m}}^{′} ( T - ξ ; \underline{θ} ) \underline{z} ( ξ ) ⅆ ξ 〉}^{2}}{\frac{ {σ}_{ n}^{ 2}}{ 2} 〈 \int {\underline{m}}^{′} ( ξ ; \underline{θ} ) \underline{m} ( ξ ; \underline{θ} ) ⅆ ξ 〉} }_{{<}_{{H}_{o}}}^{{>}^{{H}_{1}}} {λ}_{θ} . \qquad ( 14 )$$

Thus the detection of the mass is determined, when the set threshold is exceeded. If Applicants assume (simply) that the mass can be represented by a spatio-temporal point source, then performing the prescribed convolution with s(r,tₛ)=δ(t−tₛ), Applicants have that
z(t)=G′(t)*δ(t−tₛ)≡G′(t−tₛ). (15)
In terms of the matched-field approach, if Applicants assume that the unknown parameters are the source or equivalently mass position, rₛ, then Applicants see immediately that their matching or replicant vector in the medium is given by θ′ₛ=rₛ=[xₛ yₛ]′, the position of the mass, that is, the matched filter solution is
m′(t;θ)=G′(T−t+tₒ;θₛ). (16)
Therefore, Applicants can create output SNR “power” surface and detection scheme by forming the GLRT

$${max }_{{{θ}_{-}}_{π}} P ( {\underline{θ}}_{s} ) \begin{matrix}{>}^{{H}_{1}} \\ {<}_{{H}_{o}}\end{matrix} {λ}_{θ} \\ \begin{matrix}where P ( {\underline{θ}}_{s} ) = \frac{{〈 {\underline{m}}^{′} ( T ; {\underline{θ}}_{s} ) * \underline{z} ( T ) 〉}^{2}}{〈 {\underline{m}}^{′} ( T ; {\underline{θ}}_{s} ) * \underline{m} ( T ; {\underline{θ}}_{s} ) 〉} \\ = \frac{{〈 {\underline{G}}^{′} ( T - t + {t}_{o} ; {\underline{θ}}_{s} ) * \underline{z} ( T ) 〉}^{2}}{〈 {\underline{G}}^{′} ( T ; {\underline{θ}}_{s} ) * \underline{G} ( T ; {\underline{θ}}_{s} ) 〉} .\end{matrix} \qquad ( 17 )$$

Thus, the so-called “matched-field” detector/localizer uses an assumed position, θ, and the propagation model to produce the replicant, m(t;θ). The model replicant is then convolved (correlated) with the measurement, z(T) to produce the detection statistic, P(θₛ) which is compared to the threshold, λ_{θ} to detect the presence of a mass at the pixel specified by the location parameter, θ.

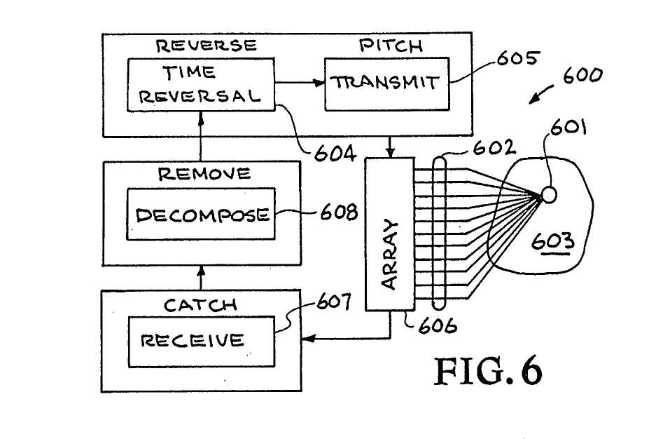

Referring now to FIG. 6, iterative time-reversal techniques are shown. The system illustrated in FIG. 6 is designated generally by the reference numeral 600. Time-reversal processing is a focusing technique that can be used to minimize the aberrations created by an inhomogeneous or random medium 603 illuminated by propagating waves 602 produced by array 606. This technique can be used to “focus” on the principal scatterer 601 dominating a pulse-echo response. The T/R technique simply processes the multichannel time series radiated from the region under investigation, collects/receive 607 the array data, decompose/digitizes 608, time-reverses 604 the temporal array signals and re-transmits 605 them back through the medium 603 to focus on each scatterer 602.

In the decoupled scatterer case, i.e., each scatterer has a distinct (fixed) eigenvalue and eigenfunction associated with it, it is possible to perform the cycle “iteratively” by focusing on the strongest mass, receiving its scattered field and removing it from the time series data, then develop an iterative scheme. The decoupling can be enhanced by introducing a small, highly scattering, reference object (a “seed”) at or near the desired point of focus. The seed becomes the strongest scatterer in the field of view of the array, enhancing the ability of the T/R technique to localize the region of interest.

The model-based focusing approach: (1) develops a model of the inhomogeneous medium including the mass under scrutiny from the results of quantitative imaging; (2) backpropagates the localized mass (source) to the array generating a set of synthesized array time series; and (3) transmits the time reversed acoustic energy back into the medium to “focus” on the target mass. In contrast to “blind” time reversal that will focus on the strongest scattering mass, the model-based approach uses the model of the medium (including the mass and its location) to synthesize the appropriate time series and focus at the correct location. Applicants apply quantitative imaging to characterize the medium model and an acoustic propagation algorithm to synthesize the required signals.

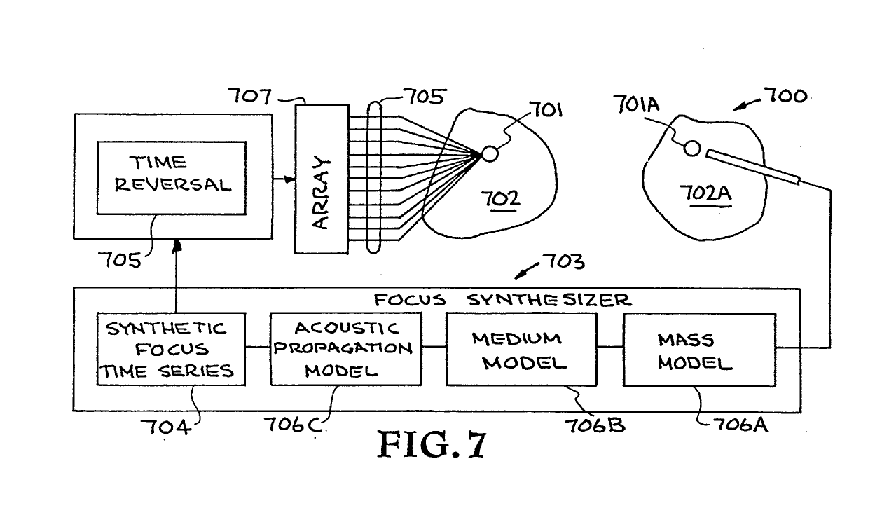

Referring now to FIG. 7, an example of interactive model-based focusing is illustrated. This example is designated generally by the reference numeral 700. Perhaps the simplest technique to localize a mass 701 under scrutiny is to enable the physician to examine the tissue image and select questionable regions for further more detailed investigations, just as a radiologist would do when examining x-rays for fractures. In this approach the physician uses, for example, an interactive light pen to select individual masses or zones requiring further detailed analysis.

A physician selects to region or zone 702 to investigate and locates the mass 701 under scrutiny providing mass position information to the focus synthesizer 703, which generates the required time series 704 that will be reversed 705 and transmitted 705 back into the tissue medium 702 by array 707. After selection of the mass 701, its position is provided as input to the focus synthesizer 703 that then generates the required time series 704 from the forward propagation/system model 706A, 706B, 706C. After reversal the focusing signals 705 are then transmitted into the medium 702 and they coherently superpose at the desired mass 701 location for treatment. Conceptually, this approach is simple, but it relies heavily on the physician to select the appropriate masses for treatment or regions to be investigated more completely.

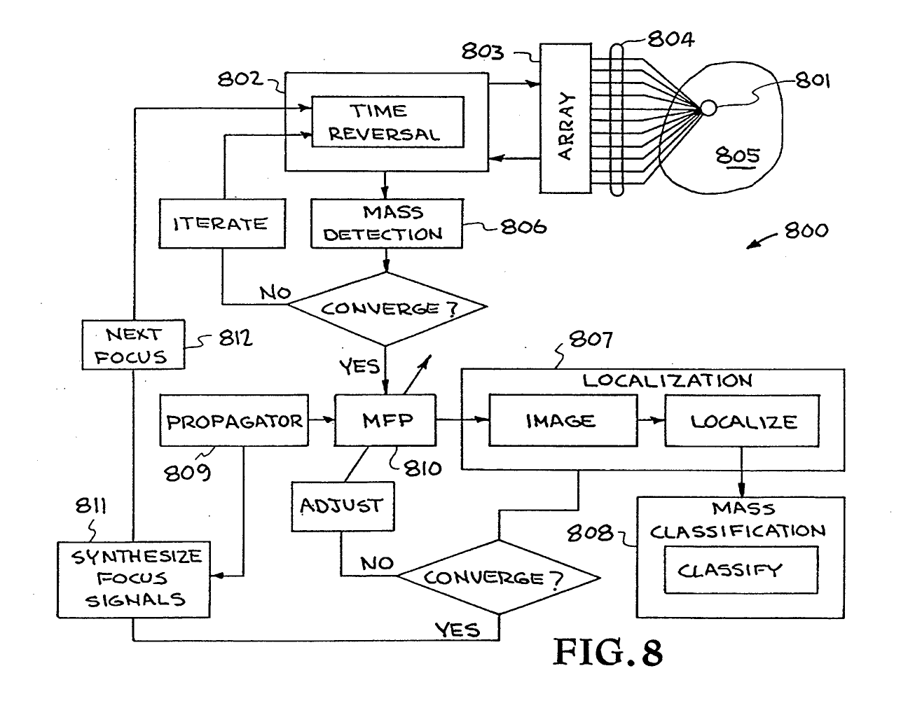

Referring now to FIG. 8, an example of model-based iterative T/R focusing is illustrated. This example is designated generally by the reference numeral 800. The example 800 combines both the strength of the iterative T/R focusing and detection capability with the model-based focus synthesizer. Here Applicants use the iterative time-reversal approach to “detect” the mass 801 in a zonal region selected by the physician. Once the mass 801 is detected, it is localized using the model-based, matched-field processor with the model developed from a quantitative image as before. After localization, the mass could be classified as benign or malignant. Once localized, the position of the mass is provided as input to the model-based focusing algorithm that produces the required set of time series. As before, the time series are reversed and transmitted into the medium to focus on the mass. After physical mass treatment, the procedure is repeated for the next mass to be treated. This approach employs the power of iterative time-reverser combined with the model-based focusing algorithms guaranteeing that the mass selected is to be treated. The algorithm of both model-based and time-reversal based offer the potential to perform noninvasive acoustic surgery.

The system 800 has the ability to noninvasively focus acoustical energy 804 generated by the array 803 in tissue 805 and directly on a tissue mass 801 such as a tumor, a cyst, etc. The system 800 comprises a time-reversal component 802, a mass detection component 806, a localization component 807, a mass classification component 808, a propagator 809, a MFP 810, a synthesize focus signals component 811, and next focus component 812. The development of a dominant mass detection algorithm using the T/R processor follows the same analysis as before using the iterative T/R models. Applicants develop a solution to the dominant mass (scatterer) detection problem. Applicants are assuming that the received field is contaminated by zero-mean, gaussian noise of variance, σᵥ², then the noisy array measurement becomes
z(r;t)=R(r;t)+V(r;t). (18)

Applicants basic problem is to determine whether Applicants have a single mass (scatterer) or equivalently has the iterative T/R processor “focused” on the dominant mass. If Applicants assume this measurement model, then Applicants must solve the following decision problem at each iteration,
H₀: zᵢ(r;t)=Vᵢ(r;t) [Noise Only]
H₁: zᵢ(r;t)=Rᵢ(r₀;t)+Vᵢ(r;t) [Signal+Noise] (19)
where zᵢ,Vᵢ,RᵢεR^{N}^{_{L}}^{×1} with the array measurement for a single scatterer defined by
Rᵢ(rₖ;t)≡gₖ(r;t)*qᵢ(rₖ;t), (20)
and qᵢ(rₖ;t) the k^{th} scatterer return (scalar) associated with the i^{th}-iteration. Also, gₖ(r;t) is an N_{L}-vector defined as the k^{th} column of the N_{L}×Nₛ-Green's function matrix. This definition can be rewritten in expanded form as

$$\begin{matrix}R ( r ; t ) = G ( r ; t ) * q ( r ; t ) \\ = [ \begin{matrix}{g}_{0} ( r ; t ) & {g}_{1} ( r ; t ) & … & {g}_{{N}_{s} - 1} ( r ; t )\end{matrix} ] * \\ [ \begin{matrix}q ( {r}_{0} ; t ) \\ q ( {r}_{1} ; t ) \\ ⋮ \\ q ( {r}_{{N}_{s} - 1} ; t )\end{matrix} ]\end{matrix} \qquad ( 21 )$$

or performing these operations, Applicants obtain

$$\begin{matrix}R ( r ; t ) = [ {g}_{o} ( r ; t ) * q ( {r}_{0} ; t ) + … + {g}_{{N}_{s} - 1} ( r ; t ) * q ( {r}_{{N}_{s} - 1} ; t ) ] \\ = {\sum }_{k = 0}^{{N}_{s} - 1} {g}_{k} ( r ; t ) * q ( {r}_{k} ; t )\end{matrix} \qquad ( 22 )$$

The solution to this problem is easily obtained from the Neyman-Pearson criterion as before in 5 given by the log-likelihood ratio test (LRT)

$$Λ ( {z}_{i} ) = ln Pr ( {z}_{i} ( r ; t ) \vert {H}_{1} ) - ln Pr ( {z}_{i} ( r ; t ) \vert {H}_{0} ) \begin{matrix}{>}^{{H}_{1}} \\ {<}_{{H}_{o}}\end{matrix} ln \tilde{λ} , \qquad ( 23 )$$

where Pr is the probability density function and \tilde{λ} is the threshold of the test. This problem, assuming that the measurements are contaminated by additive zero-mean, gaussian noise with variance σᵥ²I leads to the decision function

$$Λ ( {z}_{i} ) = - \frac{1}{2 {σ}_{v}^{2}} [ {( {z}_{i} ( r ; t ) - {R}_{i} ( r ; t ) )}^{′} ( {z}_{i} ( r ; t ) - {R}_{i} ( r ; t ) ) - {z}_{i}^{′} ( r ; t ) {z}_{i} ( r ; t ) ] \begin{matrix}{>}^{{H}_{1}} \\ {<}_{{H}_{o}}\end{matrix} ln \tilde{λ} .$$

Expanding this expression and collecting all data dependent terms, Applicants obtain the sufficient statistic

$$Λ ( {z}_{i} ) = {z}_{i}^{′} ( r ; t ) {R}_{i} ( r ; t ) \begin{matrix}{>}^{{H}_{1}} \\ {<}_{{H}_{o}}\end{matrix} {σ}_{v}^{2} ln \tilde{λ} + \frac{1}{2} {R}_{i}^{′} ( r ; t ) {R}_{i} ( r ; t ) ≡ λ .$$

Under the Neyman Pearson criterion, the threshold can be determined from the false alarm probability.

Note also that by a simple change of variables in t, it is easy to show that the sufficient statistic is the matched-filter solution with “matching” filter impulse response given in terms of Applicants vector signal model by
Rᵢ(r;T−t), and Λ(zᵢ)=Rᵢ(r;t−T)*zᵢ(r;t), (25)
which is simply the time reversed, replicant of the known field. The desired solution is to find the optimal filter at each sensor channel such that the output signal-to-noise ratio (SNR) is maximized, that is, the matched-filter is the solution

$$\begin{matrix}{max}_{{R}_{-}} SNR = \frac{{〈 {R}_{i}^{′} ( r ; T ) * {z}_{i} ( r ; T ) 〉}^{2}}{\frac{{σ}_{v}^{2}}{2} 〈 {R}_{i}^{′} ( r ; T ) * {R}_{i} ( r ; T ) 〉} \\ = \frac{{〈 \int {R}_{i}^{′} ( r ; T - ξ ) {z}_{i} ( ξ ) ⅆ ξ 〉}^{2}}{\frac{{σ}_{v}^{2}}{2} 〈 {R}_{i}^{′} ( r ; ξ ) {R}_{i} ( r ; ξ ) ⅆ ξ 〉} ,\end{matrix} \qquad ( 26 )$$

for [symbol] • [symbol] an appropriate inner product.

Applicants see that the matching or replicant vector is given by, Rᵢ(r₀;T−t), which is the time-reversed, received field induced by the dominant mass received at the array. Therefore, the detector of Eq. 25 becomes

$${P}_{i} ≡ {max}_{R} SNR = \frac{{〈 {R}_{i}^{′} ( {r}_{o} ; T ) * {z}_{i} ( r ; T ) 〉}^{2}}{\frac{{σ}_{v}^{2}}{2} 〈 {R}_{i}^{′} ( {r}_{o} ; T ) * {R}_{i} ( {r}_{o} ; T ) 〉} \begin{matrix}{>}^{{H}_{1}} \\ {<}_{{H}_{o}}\end{matrix} λ . \qquad ( 27 )$$

The problem the Applicants have now is to estimate the required replicant, Rᵢ(r₀;t), in order to implement the optimal detector. Applicants know that under certain conditions
Rᵢ(r;t) [symbol] Rᵢ(r₀;t), for i→Nᵢ,
where Nᵢ is the number of iterations required for the power method (T/R) to converge and is based on the ratio of the two largest scattering coefficients (eigenvalues). Thus, using the matched-filter theory [Joh93] developed above and the T/R focusing property, a pragmatic method of detection is to use the previous iterate, Rᵢ₋₁(r;t), produced during the “pitch-catch” sequence as the replicant and continue the iteration until the output SNR does not change, that is,

$$( \frac{{P}_{i}}{{P}_{i - 1}} ) = ( \frac{{R}_{i - 1} ( r ; T - t ) {z}_{i} ( r ; t )}{{R}_{i - 2} ( r ; T - t ) {z}_{i - 1} ( r ; t )} ) \geq T . \qquad ( 28 )$$

Clearly, Pᵢ→Pᵢ₋₁ as the T/R processor focuses on the strongest mass, that is,

$$( \frac{{P}_{i}}{{P}_{i - 1}} ) \times 100 -> 100 \% .$$

Applicants demonstrate the performance of the detector on Applicant's homogenous medium simulation and show the sequence of convolutions during the convergence of the T/R to the dominant scatterer. Here Applicants set the threshold, T=99.5% resulting in near perfect focusing and detection. Note that at each iteration the dominant mass return increases relative to the others.

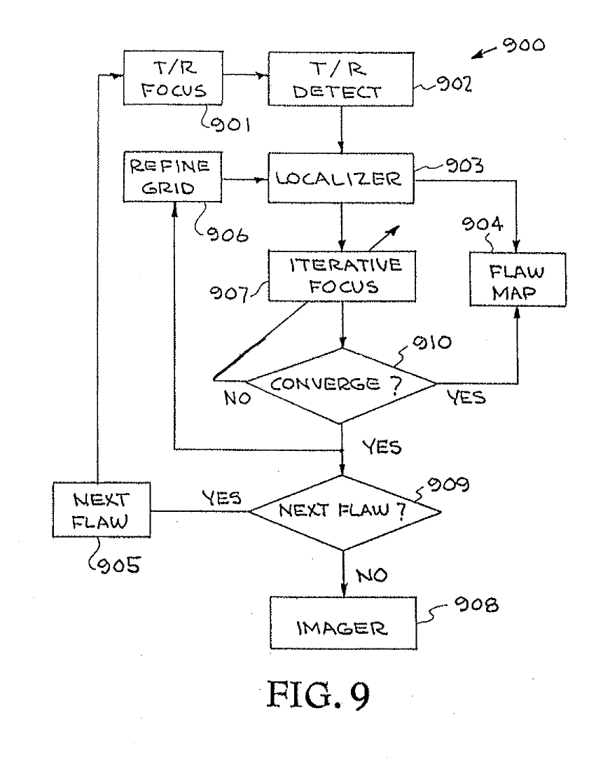

Referring now to FIG. 9, a mass localization algorithm using global/local iterations is illustrated. The algorithm is designated generally by the reference numeral 900. The elements include T/R Focus 901, T/R Detect 902, Localizer 903, Flaw Map 904, Next Flaw 905, Refine Grid 906, Iterative Focus 907, Imager 908, Next Flaw 909, and Converge 910.

Applicants developed a localization and mass detection technique (invention) based on the idea of “wave front matching.” Applicants approach is to first perform a homogeneous wave front match using a global technique to search for the best fit based on maximum power at a given location. The location (xy-position) output of this estimator then becomes the starting value for the local focusing algorithm that essentially performs a nonlinear least-squares fit over the region around the starting value. The focuser can be considered a zoom in approach to refine the grid and search. Note that it is predicated on the fact that the T/R algorithm of the previous section has focused on the strongest scatterer and the decomposition algorithm has extracted it from the total received field data. Therefore Applicants problem here is only to locate the position of this mass.

Applicants propagation model for this medium satisfies the homogeneous wave equation for a single scatterer, then under these assumptions the solution to the wave equation is that of a free space Green's function given by

$$g ( r , {r}_{o} ; t - {t}_{o} ) = \frac{δ ( t - {t}_{o} - \frac{\vert r - {r}_{o} \vert}{v} )}{4 π \vert r - {r}_{o} \vert} \qquad ( 29 )$$

with |r−rₒ|, the Euclidean distance between the source at rₒ and the observation at r.

Now returning to (28) using the homogeneous Green's function above and performing the convolution, Applicants obtain the wave field relation at the l^{th} sensor as

$$R ( {r}_{l} , t - {t}_{o} ) = \frac{1}{4 π \vert {r}_{l} - {r}_{o} \vert} s ( {r}_{o} , t - {t}_{o} - {τ}_{s} ) , \\ where {τ}_{s} = \frac{\vert {r}_{l} - {r}_{o} \vert}{v} . \qquad ( 30 )$$

If Applicants now extend these models for a single scatterer at rₒ obtained by the T/R processor over the N_{L}-element sensor array, Applicants obtain the vector relations

$$R ( {r}_{o} ; t ) = \underline{g} ( {r}_{o} ; t ) * s ( {r}_{o} ; t ) , \\ where \underline{g} ( {r}_{o} ; t ) = [ \begin{matrix}\frac{δ ( t - {τ}_{s} )}{4 π \vert {r}_{1} - {r}_{o} \vert} \\ ⋮ \\ \frac{δ ( t - {τ}_{s} )}{4 π \vert {r}_{{N}_{L}} - {r}_{o} \vert}\end{matrix} ] . \qquad ( 31 )$$

If Applicants choose to perform weighted delay-sum beam forming at the output of the array, then Applicants obtain

$$bf ( {r}_{θ} ; t ) = \frac{1}{{N}_{L}} {\sum }_{l = 1}^{{N}_{L}} {w}_{θ} ( l ) R ( {r}_{l} ; t - {t}_{o} - {τ}_{s} + {τ}_{θ} ) . \qquad ( 32 )$$

Now if the beam former is steered to the correct scatterer location, then r_{θ}=rₒ, w_{θ}(l)=4πN_{L}|rₗ−rₒ|, and τ_{θ}=tₒ+τₛ. The output is given by
bf(rₒ;t)=s(rₒ;t), (33)
and therefore, power output is maximized as
P(r_{θ})=|s(rₒ;t)|². (34)

Thus, Applicants approach to the global search technique is based on matching the homogeneous wave front that is equivalent to performing delay-sum beam forming. Let us continue with Applicants homogeneous example of the previous section and perform the following search technique:

GLOBAL SEARCH ALGORITHM (HOMOGENEOUS WAVEFRONT)
decompose the tissue dimensions into pixels (Δxᵢ,Δyⱼ),
i = 1,...,Nₓ; j = 1,...,N_{y};
$for each ( Δ {x}_{i} , Δ {y}_{j} ) calculate the corresponding time delay , {τ}_{s} ( Δ ) = \frac{\vert {\underline{r}}_{ℓ} - {\underline{r}}_{ij} \vert}{v} , Δ {x}_{i} = i Δ x , Δ {y}_{j} = j Δ y , and \vert {\underline{r}}_{ℓ} - {\underline{r}}_{ij} \vert = \sqrt{\begin{matrix}{( {x}_{ℓ} - i Δ x )}^{2} + \\ {( {y}_{ℓ} - j Δ y )}^{2}\end{matrix}} ;$
perform weighted sum-delay beam forming according to Eq. 32;
calculate the power, P(rᵢⱼ), at the array output for each pixel; and
select the pixel of maximum power as the global search position estimate.

Applicants synthesized a point mass in a homogeneous medium with sound speed 3.5 mm/usec under the same conditions of the previous example. Applicants generated the field data as before with the true synthesized mass positioned at (12 mm, 6 mm). The global search technique performs quite well (as expected) for the homogeneous case. Here Applicants see the maximum located at approximately the true position.

Once Applicants have a starting value resulting from the global search, Applicants use these estimates in a wave front matching algorithm. Applicants set up the following nonlinear least-squares problem by first defining the error between the measured receiver array outputs, R(r;t), and the estimate, \hat{R}(r;t), that is,
ε(r_{θ};t)≡R(r;t)−\hat{R}(r;t)=R(r;t)−R(r_{θ};t,\hat{θ}), (35)
which leads to the following cost function

$$J ( θ ) = \frac{1}{{N}_{L}} {ɛ}^{′} ( {r}_{θ} ; t ) ɛ ( {r}_{θ} ; t ) . \qquad ( 36 )$$

Using Eq. (28), Applicants estimate the wave front received at the array by defining the following forward propagation model, R(r;t). If Applicants have a homogeneous model, then

$$\begin{matrix}R ( r ; t , θ ) = \frac{1}{4 π {d}_{θ} ( i , j )} R ( r ; t - {τ}_{θ} ( i , j ) ) , \\ where & ( 37 ) \\ {d}_{θ} ( i , j ) = \vert r - {r}_{θ} ( i , j ) \vert and {τ}_{θ} ( i , j ) = \frac{\vert r - {r}_{θ} ( i , j ) \vert}{v} \\ for {r}_{θ} ( i , j ) = ( {x}_{i} , {y}_{j} ) . & ( 38 )\end{matrix}$$

The local focusing algorithm can be implemented by:

LOCAL SEARCH ALGORITHM (HOMOGENEOUS CASE)

initialize the search with the initial global position estimates obtained from
above, r_{θ}(i,j) = (\tilde{x}ᵢ,\tilde{y}ᵢ);
$estimate the corresponding time delays , {τ}_{θ} ( i , j ) using ( 38 ) with$ ${x}_{i} = i x , {y}_{j} = j Δ y , and \vert {r}_{ℓ} - {r}_{θ} ( i , j ) \vert = \sqrt{{( {x}_{ℓ} - i Δ x )}^{2} + {( {y}_{ℓ} - j Δ y )}^{2}} ;$
search over all {i,j}, i = 1,...,Nₓ,j = 1,...,N_{y} using the polytope
method [MAT93];
estimate for each {i,j} the mean-squared error (MSE),
J_{θ}(i,j) where ε_{θ}(i,j) ≡ R(r;t) − Rᵢⱼ(r;t,\hat{θ}); and
$select the search position estimate , {\hat{r}}_{θ} ( i , j ) = ( {x}_{i}^{*} , {y}_{i}^{*} ) corresponding to the$ $minimum MSE .$

Applicants used the same problem defined above and synthesized data at 3 dB SNR on a 32-element array driven by a narrow pulse.

One of Applicants investigations related to how well ultrasound can be used to focus in tissue. To understand this Applicants investigated the tissue composition of the breast. Breast tissue is composed of fat in which bags of connective tissue surround networks of hollow pipes or ducts lined by an extremely thin layer (1 to 2 cell) of epithelial tissue. Cancer of the breast develops in the epithelium; therefore, indicating the wide interest in imaging mammary epithelium. The anatomy of the breast shows that it consists of epithelial and connective tissue elements incorporated in an extensive system of ducts which terminate at the nipple. The ducts are surrounded by connective tissue and lined by two layers of epithelial cells. Terminal ducts communicate with the lobule, the milk secreting unit. The lobule is also composed of epithelial cells and change in size and numbers during various phases of female life cycle. Breast pathology can (simply) be considered to be comprised by three groups of lesions: focal change, fibrocystic change, and neoplasm's (tumors). Focal change lesions affect most organs such as inflammation, abscesses and hemorrhages, while fibrocystic changes evolve as cysts, duct dilatation, intraductal hyperplasia and other compound alterations. Neoplasm's are benign like intraductal papillomas or malignant including carcinomas and fibroadenoma.

Ultrasound propagation in breast tissue has ultrasonic properties of attenuation and sound velocity for various tissue types and conditions. Ultrasonic images can be used to accurately reproduce the shape and size of lesions. For example, a clear zone of low velocity (1400-1450 m/s) with low attenuation beneath the skin and external to the breast parenchyma characterizing the subcutaneous zone. The parenchyma is characterized by a pattern of intermediate velocities and attenuation. Cysts show relatively low attenuation and velocity in the range of water (1500-1525 m/s), while solid lesions in dense breasts show decreased attenuation relative to the background. Neoplasms tend to be single, more spherical in shape, and achieve the largest dimensions while variants of fibrocystic disease typically show multiple smaller regions some of which can be linear or irregular in shape. Fibrocystic disease tends to be in the central region of the breast. Extremely fibrous carcinomas tend to be high speed (>1530 m/s).

The main advantage of ultrasound is that ductal displays are always visible primarily because it is very sensitive to the physical state and mechanical properties of tissue. For instance, the elasticity and compactness determine the percentage of reflection at boundaries, while the shape and size of the boundary surface yield specular or scattered reflection. The connective tissue is described as loose, but it is made of solid collagenous fibers and behaves as a solid object well identified by ultrasound from the semiliquid fat on one side and the liquid containing ductolobular structures on the other. This property of ultrasonic interaction with breast tissue enables the display of the spatial arrangement of the fluid that fills the ductolobular structures revealing the contours of the ducts which contain the epithelium critical to cancer detection. Although the one-to-two cell layer of epithelial cells is too thin to be directly visible by current imaging system capability, the existence of occult epithelial diseases is apparent as soon as a perceptible alteration in the shape or shade of the ductolobular structures is produced. When the epithelium increases in thickness, it becomes easily observable and clearly distinguishable from the connective tissue because it shows a lower echogenicity.

When these two tissues are affected more intensely by pathologies their difference in echogenicity increases enabling the differentiation between epithelial and connective components in lesions. To summarize, the epithelium, the connective tissue and their respective pathologies are displayed in ultrasonic images by contrast enabling them to be distinguished from one another.

Applicants have used time-reversal processing to find a set of time signals along the acoustic array that are known to refocus on the small region (presumably a tumor) of interest. Then, by increasing the amplitudes of these signals (turning up the volume), the time-reversal pulse will heat the region and kill the tumor, while not causing collateral damage in the surrounding tissue. There are a number of variants on this approach to be considered.

One example of an alternative is to use model-based focusing after imaging the breast's acoustic speed distribution. Using ultrasound imaging methods developed previously, Applicants can obtain a map of the acoustic speed distribution inside the breast. When this map is input into a computer modeling code, tests can be done on how well the time-reversal focusing might proceed in the breast. Applicants then do forward modeling treating the tumor (or some central point inside the tumor) as a fictitious source. Saving the computed signal at the array locations, Applicants can use this data in two ways: (1) Do another computation that uses the time-reversed arrivals to refocus back at the point in order to determine how well T/R focusing can be achieved. (2) When satisfied that the object in question is a tumor and that sufficiently good focusing can be achieved, use the same recorded signals (originally from the simulation, but now in the actual physical array) to blast a time-reversed pulse-train back at the “tumor.” For this approach, the computational step can be viewed as a dry run, to see if it appears that the desired results can be achieved. The issue might be that with too much heterogeneity in the speed distribution, in some cases, it might not be possible to focus well enough to make the procedure viable. Then, the procedure could be terminated before doing any harm.

Exposure to ultrasound below the level of cell destruction can also increase the porosity of cell membranes to transport of therapeutic agents (chemical and genetic). In addition, focusing of ultrasound could be used to control the rupture microcapsules containing therapeutic agents. The precise control of the position and intensity of focus provided by this invention would significantly enhance the effectiveness of these techniques.

Acoustic Propagation in Breast Tissue—In comparison to the usual homogeneous wave equation (K=constant), the inhomogeneous wave equation (K is a function of position r) for propagation of a single temporal frequency signal, f, through tissue is governed physically by

$$( {∇}^{2} + {K}^{2} ( r ) ) u ( r ) = 0 , \\ {K ( r )}^{2} = {k ( r )}^{2} + \frac{1}{2 ρ ( r )} {∇}^{2} ρ ( r ) - \frac{3}{4} {( \frac{∇ ρ ( r )}{ρ ( r )} )}^{2} , \qquad ( 39 )$$

where u(r)=p(r)/\sqrt{ρ(r)}, k(r)=2πf/c(r), p(r) is the pressure, ρ(r) is the density, f is the frequency, r is the spatial position vector, and c(r) is the wave speed in the tissue. The wave speed is related to the density and bulk modulus B(r) through c(r)=\sqrt{B(r)|ρ(r)} and varies with the type of tissue in the medium. If the distribution of density and wave speed in the tissue medium can be determined then a three dimensional map of tissue types can be constructed. With this map or nonparametric model of the medium available, then focusing is a simple matter of using the forward propagation model to obtain the required time series which will be reversed to focus on the target mass as described previously as model-based focusing. The basic problem of ultrasound focusing is to determine the density and speed distributions by measuring the properties of waves launched through the tissue medium. Tissue also absorbs a portion of the sound propagating through it. This effect is often represented by a complex sound speed, c(\vec{x})=cₒ(1+ia(\vec{x})/k(\vec{x})), where cₒ is the wave speed given above and a(r) is the absorption coefficient. The value of a varies with tissue type and is another quantity that can be used to identify different tissue structures within the medium.

For breast tissue, in particular, Applicants see the variation of sound speed within the breast is approximately ±10% with fat having the slowest speed and connective tissue having the fastest speed. Fat is also the least dense tissue in the breast while connective tissue is the densest. From the relationship between sound speed and density shown above, Applicants conclude that the variation of the bulk modulus in the breast is much greater than the variation in density. Applicants can then omit the terms in the wave propagation that depend on density variation while retaining those that depend on wave speed variation to obtain

$$( {∇}^{2} + {k}^{2} ( r ) ) u ( r ) = 0 , \\ k ( r ) = \frac{2 π f}{c ( r )} . \qquad ( 40 )$$

This is the basic equation Applicants use for forward modeling of ultrasound propagation through the breast.

The problem of calculating the amplitude and phase of ultrasonic pressure waves propagating through the breast can be solved using a number of techniques applied. Various approaches have already been implemented for other problems at the Laboratory. Most of these involve the use of finite elements to represent the wave field and medium. This reduces the problem from the original partial differential equation to a matrix equation suitable for solution on a computer. The solution provides phase and amplitude at each proposed receiver around the breast. Inputs provided to the numerical model would include sound speed and absorption for each tissue type, an image or morphological description of the tissue medium and the position of each transmitter relative to the medium. Receiver phases and amplitudes can be generated for each proposed array configuration and the focusing algorithms are applied to this simulated data.

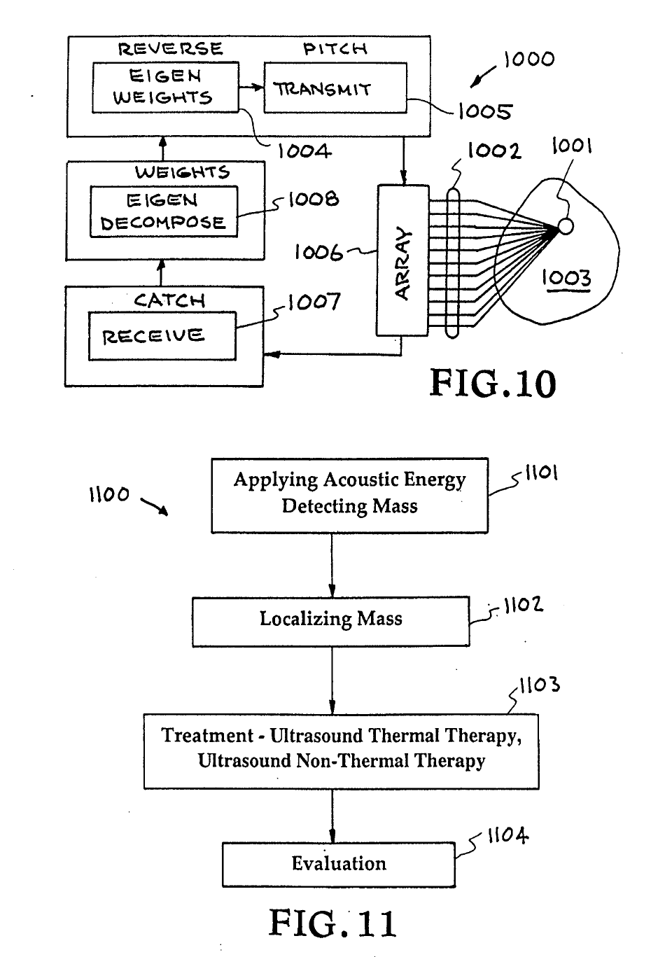

Referring now to FIG. 10, the eigen-decomposition time-reversal technique is shown. The system illustrated in FIG. 10 is designated generally by the reference numeral 1000. As we have previously mentioned, time-reversal processing is a focusing technique that can be used to minimize the aberrations created by an inhomogeneous or random medium 1001 illuminated by propagating waves 1002 produced by array 1006. The eigen-decomposition technique allows one to predetermine the number of distinguishable scatterers, select one scatterer 1003 of interest, then apply the time-reversal technique to focus on that scatterer. The technique requires transmitting a broadband pulse from each of the N array elements in sequence, collecting and storing N received signals 1007 between each transmit. The resulting N by N array (multistatic data array) of received signals is Fourier transformed and a singular value decomposition (SVD) is performed for each frequency component of interest (1008). The result is a set of singular values and singular vectors for each frequency. From each set, a particular singular vectors is selected which provides a set of eigen-weights 1004 that are used to synthesize a transmitted pulse 1005 that focuses on the selected scatterer 1003.

An alternate method of collecting the multistatic data array is to use N sets orthogonal weights, each set consisting of N individual weights, such as a Walsh basis. A broadband pulse, weighted by the N values of selected set of weights, is transmitted simultaneously by the array and the returned signals are received and recorded. This process is repeated for each set of weights, building an N by N array of received signals. Using the orthogonality of the set of weights, this N by N signal array can be transformed into the multistatic data matrix required for the eigen-decomposition technique. This alternate technique of determining the multistatic data matrix can be used to increase the signal-to-noise ratio.

The criterion used to select a particular singular vector for each frequency is determined by the user. Particular criteria may include selecting the vectors with the largest singular values for each frequency, or whose singular values fit a desired pattern as a function of frequency. Alternatively, the user may select the set of singular vectors that are close to a predetermined set of vectors, as measured by an error functional such as mean-square error. For example, if s⁽ⁿ⁾(f) is the nth singular vector for frequency f and s⁽⁰⁾(f) is a desired reference vector (normalized), the particular value of n may be determined by minimizing the mean-square error,
eₙ=∫|s⁽ⁿ⁾(f)−s⁽⁰⁾(f)|²df.
The reference vector s⁽⁰⁾(f) may be obtained using a homogeneous medium model to calculate the vector that would focus on a particular scatterer.

FIGS. 1-10 and the description above describe a system for treating tissue containing a mass to reduce or destroy the mass. The presence of a tissue mass is detected by applying acoustic energy into the tissue using an array of ultrasonic transducers. The amount of energy scattered by the mass depends on its acoustic parameters (density, sound speed, attenuation, etc.). Once it is detected, the mass is localized to determine its position within the tissue medium. When the mass is detected and localized, “zonal” focusing is performed to extract or zoom in on the tissue mass under scrutiny. Once detected and localized, temporal signatures are developed to “drive” the array and focus increased energy back onto the mass. Increased acoustic energy is transmitted back onto the mass to treat the mass and/or provide the treatment. The forms of treatment include, Ultrasound thermal therapy: hyperthermic applications, ultrasound thermal therapy: non-invasive surgery, ultrasound non-thermal therapy: controlled cavitation, and other treatments. Embodiments of the invention provide evaluation of the treatment. After the treatment, acoustic energy is propagated into the tissue using an array of ultrasonic transducers to evaluate the treatment.

Ultrasound therapy is classified by dosage parameters (i.e., field intensity and exposure time) employed during the treatment process. Generally, this classification results in two modes of operation, these are tissue susceptibility (sonothermal or sonodynamic) or tissue destruction. Tissue heating (or hyperthermia) occurs when the affected tissue is exposed to low intensity ultrasound for long periods of time typically (10-30 minutes). The resulting absorption of acoustic energy results in a localized temperature elevation in the range of (40-45° C.) for the duration of the exposure. Tissue destruction occurs when the exposed region is subjected to a sharply focused ultrasound beam for a short time typically (0.1-10 seconds). The peak intensity at the focus (300-2000 W/cm2) can elevate the tissue in the focal zone to temperatures greater than 90° C. in a few seconds. At these high temperatures, cell death occurs which results in tissue necrosis in a very short time. Outside of the focal region, where the ultrasound intensity is much lower, tissue temperature is maintained at a physiologically acceptable safe level. Thus, ultrasound therapy offers the potential of a minimally invasive surgical tool or as a mechanism to facilitate hyperthermic treatments in living tissue.

When an ultrasonic wave is launched into tissue by a transducer or an array of transducers, the wave energy is absorbed, reflected or scattered by the tissue. The reflected/scattered energy received by a transducer represents the wave interaction with the tissue and is eventually used to create the image. The reflected energy received is due to changes in acoustic impedance across interfaces, while scattering occurs when the wave interacts with structures of size comparable to or less than an acoustic wavelength.

Probably the most critical issues in ultrasonic focusing are the acoustic characteristics of the tissue. The primary characteristics to consider are sound speed, attenuation, scattering, and inhomogeneities. Sound speed in soft tissue is approximately 1500 m/s, for instance, speeds in fat are about 1410 m/s, muscle is 1566 m/s, liver is 1540 m/s, while bone is 4080 m/s. Attenuation in different tissues increases in proportion to the excitation frequency. At 1 MHz fat, muscle, liver, and bone are: 0.63, 1.3-3.3, 0.94, 20 dB/cm. Typical ultrasonic designs attempt to operate at a high frequency in order to maximize spatial resolution, since frequency is inversely proportional to wavelength (above); however, as noted, attenuation increases with frequency thereby creating the tradeoff. The acoustic impedance (impedance=density×velocity) is directly related to sound speed at an interface, thereby, controlling the amplitude of the reflected/transmitted signals. Again for these tissues (fat, muscle, liver, bone) the corresponding impedance is: 1.38, 1.7, 1.65, 7.8 10⁶ kg/m²-S. For instance, in the breast, which is dominated by fatty tissue, one of the major problems is scattering. An ultrasonic wave is scattered when it travels through tissue and the scattering pattern depends on the dimensions of the tissue structure in relation to the ultrasonic wavelength. Usually soft tissue is considered to be made up of many small scatterers which create noise in the image and must be processed to produce an enhanced image. So-called speckle noise is also a real artifact that must be reduced. Speckle is actually due to coherent illumination (and scattering) which can be reduced by broadband (in frequency) illumination. The inhomogeneity of biological tissue also distorts the ultrasonic wave because the differences in propagation speed create aberrations in the phase within the tissue.

Embodiments of Applicants invention are concerned with focusing acoustic energy within the breast in order to treat cancerous masses; therefore, we are concerned with how well ultrasound can be used to focus in tissue. To understand this we must investigate the tissue composition of the breast. Breast tissue is composed of fat in which bags of connective tissue surround networks of hollow pipes or ducts lined by an extremely thin layer (1 to 2 cell) of epithelial tissue. Cancer of the breast develops in the epithelium; therefore, indicating the wide interest in imaging mammary epithelium. The anatomy of the breast shows that it consists of epithelial and connective tissue elements incorporated in an extensive system of ducts which terminate at the nipple. The ducts are surrounded by connective tissue and lined by two layers of epithelial cells. Terminal ducts communicate with the lobule, the milk secreting unit. The lobule is also composed of epithelial cells and change in size and numbers during various phases of female life cycle. Breast pathology can (simply) be considered to be comprised by three groups of lesions: focal change, fibrocystic change, and neoplasm's (tumors). Focal change lesions affect most organs such as inflammation, abscesses and hemorrhages, while fibrocystic changes evolve as cysts, duct dilatation, intraductal hyperplasia and other compound alterations. Neoplasm's are benign like intraductal papillomas or malignant including carcinomas and fibroadenoma.

Ultrasonic images can be used to accurately reproduce the shape and size of lesions. There is a zone of low velocity (1400-1450 m/s) with low attenuation beneath the skin and external to the breast parenchyma characterizing the subcutaneous zone. The parenchyma is characterized by a pattern of intermediate velocities and attenuation. Cysts show relatively low attenuation and velocity in the range of water (1500-1525 m/s), while solid lesions in dense breasts show decreased attenuation relative to the background. Neoplasms tend to be single, more spherical in shape, and achieve the largest dimensions while variants of fibrocystic disease typically show multiple smaller regions some of which can be linear or irregular in shape. Fibrocystic disease tends to be in the central region of the breast. Extremely fibrous carcinomas tend to be high speed (>1530 m/s).

The main advantage of ultrasound is that ductal displays are always visible primarily because it is very sensitive to the physical state and mechanical properties of tissue. For instance, the elasticity and compactness determine the percentage of reflection at boundaries, while the shape and size of the boundary surface yield specular or scattered reflection. The connective tissue is described as loose, but it is made of solid collagenous fibers and behaves as a solid object well identified by ultrasound from the semiliquid fat on one side and the liquid containing ductolobular structures on the other. This property of ultrasonic interaction with breast tissue enables the display of the spatial arrangement of the fluid that fills the ductolobular structures revealing the contours of the ducts which contain the epithelium critical to cancer detection. Although the one-to-two cell layer of epithelial cells is too thin to be directly visible by current imaging system capability, the existence of occult epithelial diseases is apparent as soon as a perceptible alteration in the shape or shade of the ductolobular structures is produced. When the epithelium increases in thickness, it becomes easily observable and clearly distinguishable from the connective tissue because it shows a lower echogenicity.

Hyperthermia methods rely on directing acoustic energy into a treatment area with the goal of heating the selected tissue region to temperatures ranging from (40-46° C.) for extended periods of time, up to several hours. Hyperthermia in the 40-46° C. range can significantly enhance clinical responses to radiation therapy and has the potential for enhancing other therapies, such as chemotherapy, immuno-therapy and gene therapy. The biological rationale for each of these ultrasound-drug synergisms is twofold. First, hyperthermia is a tissue sensitizer. Pre-sensitized tissue is significantly more susceptible to the cytotoxic effect of the various radio-, chemo-, or immuno-therapies. Second, hyperthermia is in itself cytoxic by altering the local cell bio-chemical processes. This complicates the treatment process due to the fact that there will be an equivalent increase of cytotoxic effects in surrounding healthy tissue. Ultrasound technology has significant advantages that allow for a higher degree of spatial and dynamic control of heating (such as beamforming and more recently time-reversal focusing) compared to other commonly utilized heating modalities. Whether by thermal or by sonodynamic processes, controlled focused ultrasound offers significant advantages to enhancing the ultrasound-drug synergy for anticancer treatments.

There are two basic mechanisms that result in tissue damage using HIFU. The first is thermal ablation whereby localized cell death (necrosis) in the exposed tissue is due primarily from elevated temperatures (>90 C). The second is a mechanical destruction due to cavitation. Natural cavitation, in a pure fluid, is brought about by the rupture of the liquid (tensile stress failure) due to the negative pressure cycle of an acoustic signal. When the magnitude of an acoustic wave exceeds the local hydrostatic pressure cavitation will occur.

Under conditions of natural nucleation, cavitation is difficult to produce except in gas bearing tissues such as the lung or liver. Nuclei are particularly sparse in regions in non aerated tissues such as the breast, brain and heart muscle. Although sufficiently high amplitude ultrasound pulses will reliably cavitate these tissues it is secondary to the thermal heating effects. By introducing impurities, (nucleation sites) such as contrast agents into these tissues it is possible to drastically reduce the cavitation threshold below where the thermal effects are dominant. These techniques are a non-thermal ultrasound therapy where cavitation is the driving mechanism. Once cavitation has initiated, the effects can be significant. Cavitation can produce a range of effects such as sonoporation of the cell walls (useful for drug enhancement and delivery) to cell lysis and homogenization of tissue. Thermal coagulation is the process whereby direct absorption of the focused acoustic energy in the tissue results in localized elevated temperatures and non-thermal based approaches whereby the destructive mechanism is due either to localized cavitation.

Applicants use time-reversal acoustics to improve upon currently available techniques that use more traditional ways of focusing by array processing through (assumed) homogeneous acoustic propagation media. Traditional focusing is limited in part because the computations require a detailed knowledge of the propagation medium, but this detailed knowledge is seldom if ever available. In the absence of this information, the assumption must be made that the medium is approximately homogeneous in its wave speed so that the focusing calculations can be carried through. Time-reversal ultrasound processing is a completely different approach that uses experimental means to focus the beam. By actively insonifying the region of interest and then recording the signals returned to the transducers, it is possible to obtain a focused beam iteratively. By time reversing the received signal repeatedly, the array output converges on a so-called eigenfunction of the scattering operator in the insonified region. This eigenfunction is associated with a single scatterer in the medium in most of the cases of interest. If this scatterer can be shown to be a cancerous tumor, then some higher amplitude ultrasound beam can be sent directly back to the tumor using the information contained in the eigenfunction. This focused return can then be used in a number of ways.

Successful focusing of ultrasound through heterogeneous media using the time-reversal concept is based on some very fundamental results in linear acoustics. When waves are linear, they can be superposed, i.e., the amplitudes of two waves passing through the same point can be added and the result is still a solution of the acoustic wave equation. This fundamental result gives rise to the very useful concept of a Green's function or impulse response function. The Green's function is itself a function of two spatial positions, the start and the end positions (source and receiver points) of the wave. Because of superposition, the Green's function is always symmetric in these two arguments, which means that if a unit source at one position causes a response g(r,r′;t) at the receiver point, then by reversing the roles a unit source at the end point will also produce a response g(r,r′;t) at the starting point. This fact is called “reciprocity” and it is the physical basis of the phenomenology that the time-reversal method exploits.

Focused heating to kill tumors: The basic idea is to use time-reversal processing to find a set of time signals along the acoustic array that are known to refocus on the small region (presumably a tumor) of interest. Then, by increasing the amplitudes of these signals (turning up the volume), the time-reversal pulse will heat the region and hopefully kill the tumor, while not causing much collateral damage in the surrounding tissue.

One example is to use model-based focusing after imaging the breast's acoustic speed distribution. Using ultrasound imaging methods developed previously for KCI, Applicants can obtain a map of the acoustic speed distribution inside the breast. When this map is input into a computer modeling code, tests can be done on how well the time-reversal focusing might proceed in the breast. Applicants then do forward modeling treating the tumor (or some central point inside the tumor) as a fictitious source. Saving the computed signal at the array locations, Applicants can use this data in two ways: (1) Do another computation that uses the time-reversed arrivals to refocus back at the point in order to determine how well T/R focusing can be achieved. (2) When satisfied that the object in question is a tumor and that sufficiently good focusing can be achieved, use the same recorded signals (originally from the simulation, but now in the actual physical array) to blast a time-reversed pulse-train back at the “tumor.” For this approach, the computational step can be viewed as a dry run, to see if it appears that the desired results can be achieved. The issue might be that with too much heterogeneity in the speed distribution, in some cases, it might not be possible to focus well enough to make the procedure viable. Then, the procedure could be terminated before doing any harm.

Ultrasonic heating, not to the point of cell destruction, might be good for boosting the effectiveness of chemical intervention. Chemical reactions generally run faster at higher temperature and diffusion of reagents should also be improved. Since the heating is noninvasive, it would not be difficult to do this as an add on to chemotherapy and the new targeted chemical approaches.

Ultrasonic heating and/or vibratory stimulation might be useful for increasing fluid production from milk ducts that are otherwise nonproductive during fluid sampling for diagnostic purposes. Such a diagnostic is ductal lavage.

In comparison to the usual homogeneous wave equation (K=constant), the inhomogeneous wave equation (K is a function of position r) for propagation of a single temporal frequency, f, signal through tissue is governed physically by

$$( {∇}^{2} + {K}^{2} ( r ) ) u ( r ) = 0 , \\ {K ( r )}^{2} = {k ( r )}^{2} + \frac{1}{2 ρ ( r )} {∇}^{2} ρ ( r ) - \frac{3}{4} {( \frac{∇ ρ ( r )}{ρ ( r )} )}^{2} , \qquad ( 3.1 )$$

where u(r)=p(r)/\sqrt{ρ(r)}, k(r)=2πf/c(r), p(r) is the pressure, ρ(r) is the density, f is the frequency, r is the spatial position vector, and c(r) is the wave speed in the tissue. The wave speed is related to the density and bulk modulus B(r) through c(r)=\sqrt{B(r)/ρ(r)} and varies with the type of tissue in the medium. If the distribution of density and wave speed in the tissue medium can be determined then a three dimensional map of tissue types can be constructed. With this map or nonparametric model of the medium available, then focusing is a simple matter of using the forward propagation model of Eq. 3.1 to obtain the required time series which will be reversed to focus on the target mass as described previously as model-based focusing. The basic problem of ultrasound focusing is to determine the density and speed distributions by measuring the properties of waves launched through the tissue medium. Tissue also absorbs a portion of the sound propagating through it. This effect is often represented by a complex sound speed, c(\vec{x})=cₒ(1+ia(\vec{x})/k(\vec{x})), where cₒ is the wave speed given above and a(r) is the absorption coefficient. The value of a varies with tissue type and is another quantity that can be used to identify different tissue structures within the medium.

For breast tissue, in particular, the variation of sound speed within the breast is approximately ±10% with fat having the slowest speed and connective tissue having the fastest speed. Fat is also the least dense tissue in the breast while connective tissue is the densest. From the relationship between sound speed and density shown above, Applicants conclude that the variation of the bulk modulus in the breast is much greater than the variation in density. Applicants can then omit the terms in the wave propagation Eq. 3.1 that depend on density variation while retaining those that depend on wave speed variation to obtain

$$( {∇}^{2} + {k}^{2} ( r ) ) u ( r ) = 0 , \\ k ( r ) = \frac{2 π f}{c ( r )} . \qquad ( 3.2 )$$

This is the basic equation Applicants use for forward modeling of ultrasound propagation through the breast.

The problem of calculating the amplitude and phase of ultrasonic pressure waves propagating through the breast can be solved using a number of techniques applied to Eq. 3.2. Various approaches have already been implemented for other problems at the Laboratory. Most of these involve the use of finite elements to represent the wave field and medium. This reduces the problem from the original partial differential equation to a matrix equation suitable for solution on a computer. The solution provides phase and amplitude at each proposed receiver around the breast. Inputs provided to the numerical model would include sound speed and absorption for each tissue type, and the position of each transmitter relative to the medium. Receiver phases and amplitudes can be generated for each proposed array configuration and the focusing algorithms are applied to this simulated data. The first step in any focusing procedure is to insonify the medium and collect all of the sensor array data to detect and localize any potential target masses.

Tomography literally means “slice” or cross-sectional imagery. In this multi-dimensional world, an object is reconstructed from data gathered by integration along hyperplanes intersecting it. In two dimensions (2D), the hyperplane degenerates to line integrals, while three dimensional (3D) objects can be investigated in two ways: (1) as a stack of 2D slices (sometimes referred to 2.5D imaging), or (2) in its natural 3D representation. Computerized tomography (CT) refers to the use of a computer in creating a tomogram or picture of a slice. In medicine, a tomogram is simply the display of a cross section of the body at a prescribed location with a desired orientation. An arbitrary function representing properties of a cross-section could be recovered from a complete set of its projections. Thus, tomographic imaging deals with reconstructing an image from its projections, where a projection is the integral of the object in a specified angular direction. Simply speaking, a projection is the information derived from transmitted energy when an object is illuminated at a particular angle. Just how this energy propagates through the object (or at least Applicants assumption of the underlying propagation) dictates what particular tomographic reconstruction algorithm is required. In order to achieve an “optimal” solution more must be known about the object and how it is characterized. What this all means is that the more known about how sound (acoustical energy) propagates within the tissue medium, the better Applicants can design Applicants algorithms to take advantage of this knowledge and improve upon the final image.

When the sizes of the inhomogeneities are smaller than a wavelength and scattering is weak, then geometric optics or the ray theory approximations (straight-ray reconstructions) are no longer valid and therefore, wave propagation and diffraction phenomena must be considered. Diffraction tomography is essentially replacing straight ray approximations with wave propagation relations. In practice DT is very similar to transmission tomography, with the so-called Fourier Diffraction Theorem replacing the Fourier Projection-Slice Theorem. The Slice Theorem states that the Fourier transform of a projection gives the values of the 2D Fourier transform along a straight line, while the Diffraction Theorem states that a projection yields the Fourier transform over a semicircular arc in 2D Fourier space.

Acoustical imaging problems fall into three categories that are determined by the physical properties of both the object being imaged and the acoustic radiation being used to insonify the object. Applicants will refer to these three cases as: low scattering (LS), weak scattering (WS) and high scattering (HS). The LS case is one in which the straight-ray approximation is very good. Typically this is when refractive index (real part) variations are small and the wavelength is much smaller than the detector resolution and/or the effective source size, and is therefore smaller than the resolvable features in the object. The HS case occurs when there is significant diffraction and/or features with large refractive index variation within the object. Most importantly, the HS case is characterized by multiple scattering events; when each radiation quantum (photon, phonon, etc.) on average undergoes several scattering events before reaching the detector.

In one embodiment, Applicants use the DT approach for the reasons mentioned in the introduction aimed primarily at focusing energy for mass treatment not high resolution full-field imaging. Of course, it is assumed that the high resolution image is available for diagnosis, detection and localization of masses in the global region.

Diffraction tomography algorithms evolve from the basic inhomogeneous wave equation of Eq. 3.1 above which can be decomposed into a homogenous and inhomogeneous part. Applicants start with the inhomogeneous equation as
(∇²+k²)u(r)=kₒ²f(r) (3.3)
with u(r) the scalar pressure-field as before and f(r) the forcing function which depends on both the object inhomogeneities and the wave field, and kₒ=2πf/cₒ is the constant complex wave number calculated from the average properties of the inhomogeneous medium. The simplest form for the forcing function is given by
f(r)=└1−n²(r)┘u(r)=o(r)u(r) (3.4)
where the object is characterized by
o(r)=└1−n²(r)┘ (3.5)
and n is the complex index of refraction at position r given by

$$n ( r ) = \frac{{c}_{o}}{c ( r )} \qquad ( 3.6 )$$

for cₒ the sound speed in the medium and c(r) the sound speed at location r of the object.

When an object is immersed in a medium, the total field at any location can be modeled as the superposition of the incident field, uᵢ(r), and the scattered field, uₛ(r), that is,
u(r)=uᵢ(r)+uₛ(r) (3.7)

Applicants assume that the incident field is present without any inhomogeneities, that is, it satisfies
(∇²+kₒ²)uᵢ(r)=0 (3.8)

The scattered field component is assumed to be that part of the total field that can be identified solely with the inhomogeneities. Now substituting Eq. 3.7 for the total field, multiplying and using Eq. 3.8, Applicants obtain the wave equation for the scattered component as
(∇²+k²)uₛ(r)=kₒ²f(r) (3.9)
which still cannot be solved for uₛ(r) directly. However, a solution can be written using superposition in terms of the Green's function. Green's functions are used primarily to solve the wave propagation equations with forcing functions or equivalently sources. The propagation is assumed to take place in a homogeneous medium as Applicants problem of Eq. 3.8. The Green's function solution of
(∇²+kₒ²)g(r,r′)=−δ(r−r′) (3.10)
describes the fields radiated from a single point source in a homogeneous medium at r′ and g(r,r′)→g(r−r′). The forcing function can be considered an array of point scatterers composing the entire object and therefore Applicants can write it as the superposition integral
f(r)=∫f(r′)δ(r−r′)dr′

Since the forcing function in Eq. 3.10 represents a point inhomogeneity, the Green's function can be considered the field response from a single point scatterer. Because the wave equation is linear, then through superposition Applicants can sum the scattered fields resulting from each individual point scatterer, that is,
uₛ(r′)=∫g(r−r′)f(r′)dr′ (3.11)

Since the forcing function is the product of the object spatial distribution and the total field (see Eq. 3.4), Applicants still must solve this equation for the scattered field. One way to achieve this is to use the first Born approximation which is defined by substituting Eq. 3.7 into Eq. 3.11 using the definition of the forcing function to give
uₛ(r)≅u_{b}(r)=∫g(r−r′)o(r′)uᵢ(r′)dr′+∫g(r−r′)o(r′)uₛ(r′)dr′
but if the scattered field is small compared to the incident then the second integral can be ignored and the first Born approximation is given by
u_{b}(r)=∫g(r−r′)o(r′)uᵢ(r′)dr′ for uₛ<<uᵢ (3.12)

It will be shown subsequently that this relation can be used to develop the Fourier diffraction theorem analogous to the Fourier slice theorem for straight ray (geometric optics) propagation models. Applicants will restrict Applicants discussion to the 2D case. Using Eq. 3.12 Applicants assume that the object is illuminated by an incident plane wave. The corresponding 2D Green's function is given by the zero order Hankel function of the first kind

$$g ( r - {r}^{′} ) = \frac{j}{4} {H}_{o} ( k \vert r - {r}^{′} \vert ) \qquad ( 3.13 )$$

Substituting into Eq. 3.12 Applicants obtain

$${u}_{b} ( r ) = \frac{j {k}^{2}}{4} {\int \int }_{S } {H}_{o} ( k \vert r - {r}^{′} \vert ) o ( {r}^{′} ) {u}_{i} ( {r}^{′} ) ⅆ {r}^{′} \qquad ( 3.14 )$$

with S any area in the xy-plane enclosing the object cross-section. Using the plane wave decomposition of the Hankel function Applicants can write Eq. 3.14 as

$${u}_{b} ( r ) = \frac{j {k}^{2}}{4} {\int \int }_{S } o ( {r}^{′} ) {u}_{i} ( {r}^{′} ) {\int }_{- ∞}^{∞} \frac{1}{β} {ⅇ}^{j [ α ( x - {x}^{′} ) + β \vert y - {y}^{′} \vert ]} ⅆ α ⅆ {r}^{′} \qquad ( 3.15 )$$

where β=\sqrt{k²−α²}. Next Applicants assume that the incident plane wave is along the positive y-axis, uᵢ(0,y)=e^{jky} and that the scattered field is measured by a line array at y=l>y′. In this case Eq. 3.15 becomes

$${u}_{b} ( x , ℓ ) = \frac{j {k}^{2}}{4 π} {\int }_{- ∞}^{∞} ⅆ α \underbrace{{\int \int }_{S } \frac{o ( {x}^{′} , {y}^{′} )}{β} {ⅇ}^{j [ α ( x - {x}^{′} ) + β \vert ℓ - {y}^{′} \vert ]} ⅆ {x}^{′} ⅆ {y}^{′}}_{O ( α , β )}$$

but the inner integral can be written as the 2D Fourier transform, O(α,β), of the object after grouping some of the terms appropriately, that is,

$${u}_{b} ( x , ℓ ) = \frac{j {k}^{2}}{4 πβ} {\int }_{- ∞}^{∞} ⅆ {αⅇ}^{j [ α x + β ℓ ]} {\int \int }_{S } o ( {x}^{′} , {y}^{′} ) {ⅇ}^{- j [ α {x}^{′} + ( β - k ) {y}^{′} ]} ⅆ {x}^{′} ⅆ {y}^{′} \qquad ( 3.17 )$$

or simply where

$${u}_{b} ( x , ℓ ) = \frac{j {k}^{2}}{4 π β} {\int }_{- ∞}^{∞} ⅆ {αⅇ}^{j [ α x + β ℓ ]} O ( α , β - k ) \qquad ( 3.18 )$$

Taking the 1D Fourier transform of u_{b} along x, Applicants obtain

$$\begin{matrix}{U}_{b} ( ω , ℓ ) = \frac{j {k}^{2}}{4 πβ} {\int }_{- ∞}^{∞} ⅆ {αⅇ}^{jβℓ} O ( α , β - k ) {\int }_{- ∞}^{∞} {ⅇ}^{j ( ω - x )} ⅆ x \\ = \frac{j {k}^{2}}{4 πβ} {\int }_{- ∞}^{∞} {ⅇ}^{j βℓ} O ( α , β - k ) 2 πδ ( ω - x ) ⅆ α\end{matrix}$$

Applying the sifting property of the delta function and substituting for β from Eq. 3.15, Applicants obtain the desired result

$${U}_{b} ( ω , ℓ ) = \frac{j {k}^{2}}{4 π \sqrt{{k}^{2} - {ω}^{2}}} {ⅇ}^{j \sqrt{{k}^{2} - {ω}^{2}} ℓ} O ( ω , \sqrt{{k}^{2} - {ω}^{2}} - k ) \\ for \vert ω \vert < k \qquad ( 3.19 )$$

Varying [symbol] [symbol] from −k to +k, the coordinates (ω,\sqrt{k²−ω²}−k) map out a semi-circular arc in the (kₓ,k_{y})-plane. Thus, if Applicants take the 1D Fourier transform of the scattered data with an incident plane wave propagating along the +y axis then for | [symbol] |<k the transform gives values of the 2D Fourier transform of the object on a semi-circular arc with endpoints at a distance of \sqrt{2}k from the origin and zero outside.

The importance of the Fourier Diffraction Theorem is that if an object is illuminated by plane waves in many directions over 360 degrees, the resulting circular arcs in the (kₓ,k_{y})-plane fill the 2D frequency domain. The function, o(x,y), may then be reconstructed by Fourier inversion. To understand this reconstruction process, Applicants start with the scattered field (under weak scattering assumptions) that is measured by the sensor line array. The basic idea in DT is to use the results from the FDT to reconstruct the object based on inverting its Fourier transform (FT) as,

$$o ( r ) = \frac{1}{{( 2 π )}^{n}} \int O ( k ) {ⅇ}^{ⅈ k \cdot r} ⅆ k \qquad ( 3.20 )$$

The problem is that the measurements of the FT are along circular arcs in k-space. The approach taken in DT is to transform the rectangular grid of the 2DFT to the circular arcs from the scattered data measured at the sensor line array as in Eq. 3.19. This is done by first representing the wave number vector as
k=kₒ(s−sₒ) (3.21)
for s,sₒ unit vectors,
s=(cos χ,sin χ) and sₒ=(cos φₒ,sin φₒ) (3.22)
with the transmitted plane wave at angle φₒ. Now transforming Eq. 3.20 leads to the circular arc coordinate system of (χ,φₒ). Thus, calculating the transformation jacobians and differentiating, Applicants obtain the object expression (in 2D)

$$o ( r ) = \frac{{k}_{o}^{2}}{2 ( 2 π )} {\int }_{0}^{2 π} {\int }_{0}^{2 π} \sqrt{1 - {( s \cdot {s}_{o} )}^{2}} O ( {k}_{o} ( s - {s}_{o} ) ) {ⅇ}^{j {k}_{o} ( s - {s}_{o} ) \cdot r} ⅆ χ ⅆ {ϕ}_{o} \qquad ( 3.23 )$$

which is an expression for the object in the circular arc coordinate system. The collected data are a function of the projection angle θₒ and the 1D frequency ω of the scattered field along the sensor line array. Transforming to remove the χ-integral (χ→(ω,γ)) by using the relations

$$( cos χ , sin χ ) = ( \frac{ω}{{k}_{o}} , \frac{γ}{{k}_{o}} ) and γ = \sqrt{{k}^{2} - {ω}^{2}} \qquad ( 3.24 )$$

and substituting into Eq. 3.23 yields

$$O ( r ) = \frac{1}{{k}_{o}} {\int }_{- {k}_{o}}^{{k}_{o}} \frac{1}{γ} \vert ω \vert O ( {k}_{o} ( s - {s}_{o} ) ) {ⅇ}^{j {k}_{o} ( s - {s}_{o} ) \cdot r} ⅆ ω \qquad ( 3.25 )$$

or substituting the FDT results under the Born approximation of Eq. 3.19, Applicants obtain
O(kₒ(s−sₒ))=−2jγU_{b}(ω,γ−kₒ)e^{-jγl} (3.26)

Now using a rotated coordinate system r=(ξ,η) the dot product of Eq. 3.21 can be expressed as ωξ+(γ−kₒ)η and therefore substituting this relation and Eq. 3.26, Applicants obtain the final filtered backpropagation relation in terms of the (ξ,η) coordinate system as

$$\begin{matrix}o ( r ) = \frac{j {k}_{o}}{{( 2 π )}^{2}} {\int }_{0}^{2 π} {\int }_{- ∞}^{∞} {Γ}_{{ϕ}_{o}} ( ω ) H ( ω ) {G}_{η} ( ω ) {ⅇ}^{jξω} ⅆ ωχ ⅆ {ϕ}_{o} \\ where & ( 3.27 ) \\ \begin{matrix}{Γ}_{{ϕ}_{o}} ( ω ) = {U}_{b} ( ω , γ - {k}_{o} ) {ⅇ}^{- j γℓ} , & [ Data ] \\ H ( ω ) = \{ \begin{matrix}\vert ω \vert & ω \leq {k}_{o} \\ 0 & elsewhere\end{matrix} & [ Filter ] \\ {G}_{η} ( ω ) = \{ \begin{matrix}{ⅇ}^{j ( γ - {k}_{o} ) η} & ω \leq {k}_{o} \\ 0 & elsewhere\end{matrix} & [ Propagator ]\end{matrix} & ( 3.28 )\end{matrix}$$

From these relations Applicants can observe the particular operations performed by the algorithm when implemented. Applicants see how the 1DFT of the “data” is used in conjunction with the FDT to obtain the arcs in the 2D Fourier domain. Applicants also note the “filtering” function evolving from the transformation of coordinates and finally the “propagator” which when convolved with the filter provides the “backpropagation” part of the algorithm. Note that this is just the theoretical basis. Other more efficient algorithms have been and will continue to be developed in the future.

The ability to detect a mass (scatterer) or multiple masses (scatterers) covers a broad spectrum of applications ranging from the detection and destruction of painful kidney or gall stones to non-invasive surgery for mass treatment proposed herein. All of these applications have one common thread—they are based on a pulse-echo principle for detection. Here the applications are usually concerned with detection, imaging and sometimes destruction (biomedical) of the reflective source (mass, stone etc.) for acoustic surgery. In these types of systems, a piezoelectric transducer first transmits a short transient pulse and then detects the echoes received back from the various scatterers similar to a radar system designed to detect and track targets.

Applicants are concerned with dynamic focusing of acoustic energy to treat tissue masses while minimizing collateral damage. Conceptually, Applicants propose a methodology based on the dynamic focusing concept called “time-reversal (T/R) focusing.” This nomenclature has evolved recently (early 1990's) from the optics area where time-reversal is the dynamic broadband analog of the well-known phase conjugate mirror (PCM) used to focus narrowband monochromatic waves. Thus, in concept, the T/R mirror can be thought of as a broadband version of a PCM. This same basic reversal principle holds in digital signal processing in two-pass digital filter design in which a signal is filtered, reversed and re-filtered to provide an enhanced signal with the phase preserved indicating a zero-phase filter response. In fact, from the signal processing perspective T/R focusing represents the “optimal” spatio-temporal matched filter in the sense of maximizing the output signal-to-noise ratio (SNR).

Time-reversal processing is a focusing technique which can be used to eliminate the aberrations created by an inhomogeneous or random medium illuminated by propagating waves. This technique can be used to “focus” on the principal scatterer dominating a pulse-echo response. The applicability of time-reversal processing to focus energy without the need to model the medium is a tantalizingly important property, since most media are unknown and random (in the worst case) and frankly temporal coherence (time delay) processing no longer is applicable. A T/R technique simply processes the multichannel time series radiated from the region under investigation, collects the array data, digitizes, time-reverses the temporal array signals and re-transmits them back through the medium to focus on each scatterer. Thus, this proposal is on the cutting edge of the current research and could lead to new frontiers in the biomedical applications areas.

The basic principle of time-reversal processing, in its simplest form can succinctly be characterized by the following. Consider the spatio-temporal propagation of a source, s(rₒ,t) located at rₒ and time t through a medium characterized by the Green's function (impulse response) G(r,rₒ;t) from the source to location r. From systems theory Applicants know that this operation is given by convolution to yield the received signal, that is,
R(r,t)=G(r,rₒ;t)*s(rₒ,t) [symbol] R(r,ω)=G(r,rₒ;ω)S(rₒ,ω), (3.29)
where for simplicity Applicants assume a unity scattering coefficient. Applicants have also included the equivalent Fourier transform representation. Based on the underlying theory, Applicants “re-transmit” or “back-propagate” from r, through the medium, back to the original source position at rₒ, and Applicants choose to transmit the time-reversed signal, R(r,−t), as depicted in 10b, then the Applicants have that
ŝ(rₒ,t)=G(rₒ,r;t)*R(r,−t) [symbol] Ŝ(rₒ,ω)=G(rₒ,r;ω)R*(r,ω), (3.30)
utilizing the Fourier transform conjugation property. But substituting the reversed signal into Eq. 3.30 and invoking the Reciprocity Theorem (G(rₒ,r;t)≡G(r,rₒ;t)) interchanging source and receiver position, Applicants obtain
ŝ(rₒ,t)=G(rₒ,r;t)*G(rₒ,r;−t)*s(rₒ,−t) [symbol] Ŝ(rₒ,ω)=|G(r,rₒ;ω)|²S*(rₒ,ω), (3.31)
which implies that the reversed signals re-transmitted through the medium will “focus” the enhanced energy (with gain K) back to the original source position with no change in phase (FIG. 9c) because of the magnitude-squared Green's function, that is,
Ŝ(rₒ,ω)∝KS*(rₒ,ω), (3.32)
precisely demonstrating the broadband version of phase conjugation. Clearly, this relation is more complicated, and more sophisticated representations including sensor transfer functions, noise, etc. can be included, but the underlying T/R principle remains invariant—the phase has not been altered and the reversed signal re-focuses back to the original source location! Knowledge of the Green's function is not required (no modeling). The T/R operator is merely a focuser much like adjusting the focus in a telescope. This simple property can be extended to random media, since the T/R signal returns to the source along the same path it was originally transmitted.

Referring now to FIG. 11, a conceptual illustration of a system for noninvasive mass treatment and evaluation is shown. The system is designated generally by the reference numeral 1100. The system 1100 comprises apparatus and method for treating a mass within tissue by transmitting and receiving acoustic signals from the tissue with a plurality of acoustic detectors; applying treatment to the mass, wherein the step of applying treatment to the mass comprises directing acoustic radiation to the mass; and evaluating the effect of the treatment on the mass by receiving acoustic signals scattered from the tissue with a plurality of acoustic detectors. That system can be described as a set of four steps.

First as illustrated by block 1101, Applicants detect the presence of a tissue mass applying acoustic energy propagated into the tissue using an array of ultrasonic transducers. The amount of energy scattered by the mass depends on its acoustic parameters (density, sound speed, attenuation, etc.).

Second as illustrated by block 1102, once it is detected, the mass is localized to determine its position within the tissue medium. When the mass is detected and localized, “zonal” focusing is performed to extract or zoom in on the tissue mass under scrutiny. Once detected and localized, temporal signatures are developed to “drive” the array and focus increased energy back onto the mass.

Third as illustrated by block 1103, after it is decided to treat the mass, increased acoustic energy is transmitted back onto the mass to provide the treatment. The forms of treatment include, Ultrasound thermal therapy: hyperthermic applications, Ultrasound thermal therapy: non-invasive surgery, Ultrasound non-thermal therapy: controlled cavitation, and other treatments.

Fourth as illustrated by block 1104, after the treatment acoustic energy propagated into the tissue using an array of ultrasonic transducers to evaluate the treatment.

In some embodiments, the step of receiving acoustic signals scattered from the tissue provides information derived from the received acoustic signals and the step of applying treatment to the mass comprises focusing acoustic radiation into the mass in accordance with the information derived from the received acoustic signals. The step of focusing acoustic radiation into the mass is accomplished by applying time reversal. One embodiment includes the step of determining a focal point with an object proximate the tissue. One embodiment includes the step of depositing an acoustically reflective seed into the tissue. In one embodiment the step of applying treatment to the mass comprises sonoporating at least a portion of the tissue. In one embodiment the step of applying treatment to the mass comprises delivering chemotherapy to the mass by delivering microbubbles containing the chemotherapy to the location of the mass; and damaging the microbubbles to release the chemotherapy. In one embodiment the step of damaging the microbubbles comprises focusing acoustic radiation on the microbubbles. In one embodiment the step of applying treatment to the mass comprises delivering a genetic agent to the mass. In one embodiment the step of delivering a genetic agent to the mass comprises focusing acoustic radiation on the genetic agent.

One embodiment of Applicants invention provides a method of noninvasively focusing acoustical energy on a mass within a substance to reduce or eliminate the mass. The presence of the mass in the substance is detected by applying acoustic energy to the substance. The mass is localized to determine its position within the substance. Temporal signatures are developed to drive the acoustical energy on the mass. Dynamic focusing of the acoustical energy on the mass in the substance to reduce or eliminate the mass is accomplished utilizing the temporal signatures. In one embodiment the dynamic focusing of the acoustical energy on the mass utilizes time reversal. In another embodiment, the focusing of acoustical energy on a mass utilizes modeling and time reversal. In another embodiment, the focusing of acoustical energy on a mass utilizes modeling.

In one embodiment, Applicants invention provides a method of treating tissue by noninvasively focusing acoustical energy on a mass within the tissue to reduce or eliminate the mass. The embodiment comprising the steps of detecting the presence of the mass in the tissue by applying acoustic energy to the tissue, localizing the mass to determine its position within the tissue, developing temporal signatures to drive the acoustical energy on the mass, and dynamic focusing the acoustical energy on the mass in the tissue utilizing the temporal signatures to reduce or eliminate the mass. In one embodiment, the step of dynamic focusing the acoustical energy on the mass utilizes time reversal. In another embodiment the step of step of dynamic focusing the acoustical energy on the mass utilizes modeling and time reversal. In another embodiment the step of step of dynamic focusing the acoustical energy on the mass utilizes modeling.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of noninvasively focusing acoustical energy on a mass within a substance to reduce or eliminate said mass, comprising the steps of:
detecting the presence of said mass in said substance by applying acoustic energy to said substance,
localizing said mass to determine its position within said substance,
developing temporal signatures to drive said acoustical energy on said mass, and
dynamic focusing said acoustical energy on said mass in said substance utilizing said temporal signatures to reduce or eliminate said mass.

2. The method of noninvasively focusing acoustical energy on a mass of claim 1 wherein said step of dynamic focusing said acoustical energy on said mass utilizes time reversal.

3. The method of claim 2 including identifying a point of interest within said substance and placing a small seed at said point of interest to enhance said time reversal.

4. The method of noninvasively focusing acoustical energy on a mass of claim 1 wherein said step of dynamic focusing said acoustical energy on said mass utilizes modeling and time reversal.

5. The method of noninvasively focusing acoustical energy on a mass of claim 1 wherein said step of step of dynamic focusing said acoustical energy on said mass utilizes modeling.

6. The method of noninvasively focusing acoustical energy on a mass of claim 1 wherein said step of detecting the presence of said mass in said substance comprises transmitting an initial acoustic signal into said substance for detecting said mass and detecting said initial acoustic signal.

7. The method of noninvasively focusing acoustical energy on a mass of claim 6 wherein said step of developing temporal signatures to drive said acoustical energy on said mass comprises digitizing said initial acoustic signal and time-reversing said digitized initial acoustic signal.

8. The method of noninvasively focusing acoustical energy on a mass of claim 7 wherein said step of dynamic focusing said acoustical energy on said mass in said substance comprises using said time-reversed initial acoustic signal in focusing said acoustical energy on said mass in said substance.

9. The method of noninvasively focusing acoustical energy on a mass of claim 1 wherein said step of detecting the presence of said mass in said substance comprises applying acoustic energy propagated into said substance using an array of ultrasonic transducers.

10. The method of noninvasively focusing acoustical energy on a mass of claim 1 wherein said step of dynamic focusing said acoustical energy on said mass in said substance utilizing time reversal generates heat.

11. The method of noninvasively focusing acoustical energy on a mass of claim 10 wherein said heat essentially cooks said mass insuring reduction or elimination of said mass.

12. The method of noninvasively focusing acoustical energy on a mass of claim 1 wherein said step of dynamic focusing said acoustical energy on said mass in said substance utilizing time reversal mechanically disrupts said mass.

13. A method of treating tissue by noninvasively focusing acoustical energy on a mass within said tissue to reduce or eliminate said mass, comprising the steps of:
detecting the presence of said mass in said tissue by applying acoustic energy to said tissue,
localizing said mass to determine its position within said tissue,
developing temporal signatures to drive said acoustical energy on said mass, and
dynamic focusing said acoustical energy on said mass in said tissue utilizing said temporal signatures to reduce or eliminate said mass.

14. The method of treating tissue of claim 13 wherein said step of dynamic focusing said acoustical energy on said mass utilizes time reversal.

15. The method of treating tissue of claim 14 including the steps of identifying a point of interest in said tissue and placing a small seed at said point of interest to enhance said time reversal.

16. The method of treating tissue of claim 13 wherein said step of step of dynamic focusing said acoustical energy on said mass utilizes modeling and time reversal.

17. The method of treating tissue of claim 13 wherein said step of step of dynamic focusing said acoustical energy on said mass utilizes modeling.

18. The method of treating tissue of claim 13 wherein said step of detecting the presence of said mass in said tissue comprises transmitting an initial acoustic signal into said tissue for detecting said mass and detecting said initial acoustic signal.

19. The method of treating tissue claim 18 wherein said step of developing temporal signatures to drive said acoustical energy on said mass comprises digitizing said initial acoustic signal and time-reversing said digitized initial acoustic signal.

20. The method of treating tissue of claim 19 wherein said step of dynamic focusing said acoustical energy on said mass in said tissue comprises using said time-reversed initial acoustic signal in focusing said acoustical energy on said mass in said tissue.

21. The method of treating tissue of claim 13 wherein said step of detecting the presence of said mass in said tissue comprises applying acoustic energy propagated into said tissue using an array of ultrasonic transducers.

22. The method of treating tissue of claim 13 wherein said step of dynamic focusing said acoustical energy on said mass in said tissue utilizing time reversal generates heat.

23. The method of treating tissue of claim 22 wherein said heat essentially cooks said mass insuring reduction or elimination of said mass.

24. The method of treating tissue of claim 13 wherein said step of dynamic focusing said acoustical energy on said mass in said tissue utilizing time reversal mechanically disrupts the tissue.

25. The method of treating tissue of claim 13 wherein said step of dynamic focusing said acoustical energy on said mass in said tissue utilizing time reversal increases the porosity of the cell membranes in the tissue.

26. The method of treating tissue of claim 25 wherein said increase of cell membrane porosity enhances the uptake of chemical or genetic therapeutic agents.

27. The method of treating tissue of claim 13 wherein said step of dynamic focusing said acoustical energy on said mass in said tissue utilizing time reversal locally ruptures microcapsules containing chemical or genetic therapeutic agents.

28. A system for noninvasively focusing acoustical energy on a mass in a substance to reduce or eliminate said mass, comprising:
means for applying acoustic energy to said substance for detecting said mass,
means for localizing said mass,
means for developing temporal signatures for driving said acoustical energy, and
means for dynamic focusing said acoustical energy through said substance on said mass to reduce or eliminate said mass.

29. The system of noninvasively focusing acoustical energy on a mass of claim 28 wherein said means for dynamic focusing said acoustical energy on said mass utilizes time reversal.

30. The system of noninvasively focusing acoustical energy on a mass of claim 29 wherein a small seed is placed at the point of interest to enhance time reversal.

31. The system of noninvasively focusing acoustical energy on a mass of claim 28 wherein said means for dynamic focusing said acoustical energy on said mass utilizes modeling and time reversal.

32. The system of noninvasively focusing acoustical energy on a mass of claim 28 wherein said means for dynamic focusing said acoustical energy on said mass utilizes modeling.

33. The system of noninvasively focusing acoustical energy on a mass of claim 28 wherein said means for detecting the presence of said mass in said substance comprises transmitting an initial acoustic signal into said substance for detecting said mass and detecting said initial acoustic signal.

34. The system of noninvasively focusing acoustical energy on a mass of claim 33 wherein said means for developing temporal signatures to drive said acoustical energy on said mass comprises digitizing said initial acoustic signal and time-reversing said digitized initial acoustic signal.

35. The system of noninvasively focusing acoustical energy on a mass of claim 34 wherein said means for dynamic focusing said acoustical energy on said mass in said substance comprises using said time-reversed initial acoustic signal in focusing said acoustical energy on said mass in said substance.

36. The system of noninvasively focusing acoustical energy on a mass of claim 28 wherein said means for detecting the presence of said mass in said substance comprises applying acoustic energy propagated into said substance using an array of ultrasonic transducers.

37. The system of noninvasively focusing acoustical energy on a mass of claim 28 wherein said means for dynamic focusing said acoustical energy on said mass in said substance utilizing time reversal generates heat.

38. The system of noninvasively focusing acoustical energy on a mass of claim 37 wherein said heat essentially cooks said mass insuring reduction or elimination of said mass.

39. The system of noninvasively focusing acoustical energy on a mass of claim 28 wherein said step of dynamic focusing said acoustical energy on said mass in said tissue utilizing time reversal mechanically disrupts the tissue.

40. The system of noninvasively focusing acoustical energy on a mass of claim 28 wherein said step of dynamic focusing said acoustical energy on said mass in said tissue utilizing time reversal increases the porosity of the cell membranes in the tissue.

41. The system of noninvasively focusing acoustical energy on a mass of claim 40 wherein said increase of cell membrane porosity enhances the uptake of chemical or genetic therapeutic agents.

42. The system of noninvasively focusing acoustical energy on a mass of claim 28 wherein said step of dynamic focusing said acoustical energy on said mass in said tissue utilizing time reversal locally ruptures microcapsules containing chemical or genetic therapeutic agents.

43. A system for treating tissue by treating tissue within said tissue to reduce or eliminate said mass, comprising:
means for applying acoustic energy to said substance for detecting said mass,
means for localizing said mass,
means for developing temporal signatures for driving said acoustical energy, and
means for dynamic focusing said acoustical energy through said substance on said mass to reduce or eliminate said mass.

44. The system of treating tissue of claim 43 wherein said means for dynamic focusing said acoustical energy on said mass utilizes time reversal.

45. The system of treating tissue of claim 44 wherein a small seed is placed at the point of interest to enhance time reversal.

46. The system of treating tissue of claim 43 wherein said means for dynamic focusing said acoustical energy on said mass utilizes modeling and time reversal.

47. The system of treating tissue of claim 43 wherein said means for dynamic focusing said acoustical energy on said mass utilizes modeling.

48. The system of treating tissue of claim 43 wherein said means for detecting the presence of said mass in said substance comprises transmitting an initial acoustic signal into said substance for detecting said mass and detecting said initial acoustic signal.

49. The system of treating tissue of claim 43 wherein said means for developing temporal signatures to drive said acoustical energy on said mass comprises digitizing said initial acoustic signal and time-reversing said digitized initial acoustic signal.

50. The system of treating tissue of claim 49 wherein said means for dynamic focusing said acoustical energy on said mass in said substance comprises using said time-reversed initial acoustic signal in focusing said acoustical energy on said mass in said substance.

51. The system of treating tissue of claim 43 wherein said means for detecting the presence of said mass in said substance comprises applying acoustic energy propagated into said substance using an array of ultrasonic transducers.

52. The system of treating tissue of claim 43 wherein said means for dynamic focusing said acoustical energy on said mass in said substance utilizing time reversal generates heat.

53. The system of treating tissue of claim 52 wherein said heat essentially cooks said mass insuring reduction or elimination of said mass.

54. The system of treating tissue of claim 43 wherein said step of dynamic focusing said acoustical energy on said mass in said tissue utilizing time reversal mechanically disrupts the tissue.

55. The system of treating tissue of claim 43 wherein said step of dynamic focusing said acoustical energy on said mass in said tissue utilizing time reversal increases the porosity of the cell membranes in the tissue.

56. The system of treating tissue of claim 55 wherein said increase of cell membrane porosity enhances the uptake of chemical or genetic therapeutic agents.

57. The system of treating tissue of claim 43 wherein said step of dynamic focusing said acoustical energy on said mass in said tissue utilizing time reversal locally ruptures microcapsules containing chemical or genetic therapeutic agents.

58. A system for noninvasively focusing acoustical energy on a mass in a substance, comprising:
a detector that transmits an initial acoustic signal into said substance, detects said mass, and produces an initial acoustic signal,
a processor that digitizes said initial acoustic signal,
a time-reversal processor that converts said initial acoustic signal that has been digitized into a time-reversal signal, and
an acoustic energy device that uses said time-reversal signal and focuses said acoustical energy on said mass in said substance.

59. A method of treating a mass within tissue, comprising:
receiving acoustic signals scattered from said tissue with a plurality of acoustic detectors disposed to at least partially surround at least a portion of said tissue;
applying treatment to said mass, wherein said step of applying treatment to said mass comprises directing acoustic radiation to said mass; and
evaluating the effect of said treatment on said mass by receiving acoustic signals scattered from said tissue with a plurality of acoustic detectors.

60. The method of claim 59, wherein said step of receiving acoustic signals scattered from said tissue provides information derived from the received acoustic signals and wherein said step of applying treatment to said mass further comprises focusing acoustic radiation into said mass in accordance with said information derived from the received acoustic signals.

61. The method of claim 59, wherein said step of directing acoustic radiation comprises applying time reversal.

62. The method of claim 59, wherein said step of receiving acoustic signals scattered from said tissue provides time reversal information derived from the received acoustic signals and wherein said step of applying treatment to said mass further comprises applying time reversal and focusing acoustic radiation into said mass in accordance with said applying time reversal information derived from the received acoustic signals.

63. The method of claim 59, further comprising determining a focal point with an object proximate said tissue.

64. The method of claim 59, further comprising depositing an acoustically reflective seed into said tissue.

65. The method of claim 59, wherein said step of applying treatment to said mass comprises sonoporating at least a portion of said tissue.

66. The method of claim 59, wherein said step of applying treatment to said mass comprises delivering chemotherapy to said mass by delivering microbubbles containing the chemotherapy to the location of said mass; and damaging said microbubbles to release said chemotherapy.

67. The method of claim 66, wherein said step of damaging said microbubbles comprises focusing acoustic radiation on said microbubbles.

68. The method of claim 59, wherein said step of applying treatment to said mass comprises delivering a genetic agent to said mass.

69. The method of claim 68, wherein said step of delivering a genetic agent to said mass comprises focusing acoustic radiation on said genetic agent.

70. The method of claim 59, wherein said step of applying treatment to said mass comprises ultrasound thermal therapy.

71. The method of claim 59, wherein said step of applying treatment to said mass comprises hyperthermic applications.

72. The method of claim 59, wherein said step of applying treatment to said mass comprises non-invasive surgery.

73. The method of claim 59, wherein said step of applying treatment to said mass comprises ultrasound non-thermal therapy.

74. The method of claim 59, wherein said step of applying treatment to said mass comprises controlled cavitation.
